(19) United States
(12) Patent Application Publication
(10) Pub. No.: US 2024/0246966 A1
(43) Pub. Date: Jul. 25, 2024

(54) NEW PYRAZOLO[1,5-a] PYRIMIDINE DERIVATIVES AS SIGMA LIGANDS

(71) Applicant: ACONDICIONAMIENTO TARRASENSE, Barcelona (ES)

(72) Inventors: Adriana LORENTE-CRIVILLÉ, Barcelona (ES); Carmen ALMANSA-ROSALES, Barcelona (ES)

(21) Appl. No.: 18/555,720

(22) PCT Filed: Apr. 8, 2022
(86) PCT No.: PCT/EP2022/059439

§ 371 Date: Oct. 16, 2023

(30) Foreign Application Priority Data
Apr. 16, 2021 (EP) 21382324.8

(51) Int. Cl. C07D 471/04; A61K 31/444; A61K 31/4545; A61K 31/519; A61K 31/553; C07D 487/04; C07D 519/00

(52) U.S. Cl. CPC C07D 471/04; A61K 31/444; A61K 31/4545; A61K 31/519; A61K 31/553; C07D 487/04; C07D 519/00

(57) ABSTRACT

The present invention relates to new compounds of formula (I″):

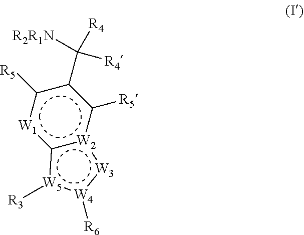

as sigma ligands having a great affinity for sigma receptors, sigma-1 receptor (σ₁) and/or sigma-2 receptor (σ₂). The present invention also refers to the process for the preparation thereof, to compositions comprising them, and to their use as medicaments.

### FIELD OF THE INVENTION

The present invention relates to new compounds of formula (I′):

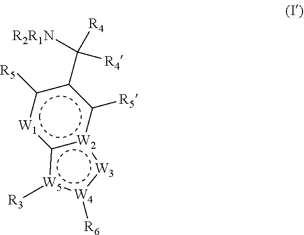

as sigma ligands having a great affinity for sigma receptors, sigma-1 receptor (σ₁) and/or sigma-2 receptor (σ₂). The present invention also refers to the process for the preparation thereof, to compositions comprising them, and to their use as medicaments.

### BACKGROUND OF THE INVENTION

The search for new therapeutic agents has been greatly aided in recent years by better understanding of the structure of proteins and other biomolecules associated with target diseases. One important class of these proteins are the sigma (σ) receptors, originally discovered in the central nervous system (CNS) of mammals in 1976 and initially related to the dysphoric, hallucinogenic and cardiac stimulant effects of opioids. Subsequent studies established a complete distinction between the o receptors binding sites and the classical opiate receptors. From studies of the biology and function of sigma receptors, evidence has been presented that sigma receptor ligands may be useful in the treatment of psychosis and movement disorders such as dystonia and tardive dyskinesia, and motor disturbances associated with Huntington's chorea or Tourette's syndrome and in Parkinson's disease [Walker, J. M. et al., Pharmacological Reviews, (1990), 42, 355]. It has been reported that the known sigma receptor ligand rimcazole clinically shows effects in the treatment of psychosis [Snyder, S. H., Largent, B. L., J. Neuropsychiatry, (1989), 1, 7]. The sigma binding sites have preferential affinity for the dextrorotatory isomers of certain opiate benzomorphans, such as (+)-SKF-10047, (+)-cyclazocine, and (+)-pentazocine and also for some narcoleptics such as haloperidol. The sigma receptor has two subtypes that were initially discriminated by stereoselective isomers of these pharmacoactive drugs. (+)-SKF-10047 has nanomolar affinity for the sigma-1 (σ₁) site and has micromolar affinity for the sigma-2 (σ₂) site. Haloperidol has similar affinities for both subtypes.

The σ₁ receptor is expressed in numerous adult mammal tissues (e.g. central nervous system, ovary, testicle, placenta, adrenal gland, spleen, liver, kidney, gastrointestinal tract) as well as in embryo development from its earliest stages, and is apparently involved in a large number of physiological functions. Its high affinity for various pharmaceuticals has been described, such as for (+)-SKF-10047, (+)-pentazocine, haloperidol and rimcazole, among others, known ligands with analgesic, anxiolytic, antidepressive, antiamnesic, antipsychotic and neuroprotective activity. Hence, the 01 receptor has possible physiological roles in processes related to analgesia, anxiety, addiction, amnesia, depression, schizophrenia, stress, neuroprotection and psychosis

[Walker, J. M. et al., Pharmacological Reviews, (1990), 42, 355; Kaiser, C. et al., Neurotransmissions, (1991), 7 (1), 1-5; Bowen, W. D., Pharmaceutica Acta Helvetiae, (2000), 74, 211-218].

The σ₁ receptor is a ligand-regulated chaperone of 223 amino acids and 25 kDa cloned in 1996 and crystallized twenty years later [Hanner, M. et al., Proc. Natl. Acad. Sci. USA, (1996), 93, 8072-8077; Su, T. P. et al., Trends Pharmacol. Sci., (2010), 31, 557-566; Schmidt, H. R. et al., Nature, (2016), 532, 527-530]. Residing primarily in the interface between the endoplasmic reticulum (ER) and mitochondrion, referred to as the mitochondria-associated membrane (MAM), it can translocate to the plasma membrane or ER-membrane and regulate the activity of other proteins by modulating N-methyl-D-aspartic (NMDA) receptors and several ion channels [Monnet, F. P. et al., Eur. J. Pharmacol., (1990), 179, 441-445; Cheng, Z. X. et al., Exp. Neurol., (2010), 210, 128-136]. Owing to the role played by the σ₁R in modulating pain-related hypersensitivity and sensitization phenomena, σ₁R antagonists have been also proposed for the treatment of neuropathic pain [Drews, E. et al., Pain, 2009, 145, 269-270; De la Puente, B. et al., Pain (2009), 145, 294-303; Díaz, J. L. et al., J. Med. Chem., (2012), 55, 8211-8224; Romero et al., Brit. J. Pharm., (2012), 166, 2289-2306; Merlos, M. et al., Adv. Exp. Med. Biol., (2017), 964, 85-107]. Additionally, the σ₁ receptor has been known to modulate opioid analgesia, and the relationship between the u-opioid and σ₁ receptors has been shown to involve direct physical interaction, which explains why σ₁ receptor antagonists enhance the antinociceptive effect of opioids without increasing their adverse effects [Chien, C. C. et al, J. Pharmacol. Exp. Ther., (1994), 271, 1583-1590; King, M. et al, Eur. J. Pharmacol., (1997), 331, R5-6; Kim, F. J. et al., Mol. Pharmacol., (2010), 77, 695-703; Zamanillo, D. et al., Eur. J. Pharmacol., (2013), 716, 78-93].

The σ₂ receptor was initially identified by radioligand binding as a site with high affinity for di-o-tolylguanidine (DTG) and haloperidol [Hellewell, S. B. et al., Brain Res., (1990), 527, 244-253]. Two decades later, progesterone receptor membrane component 1 (PGRMC1), a cytochrome-related protein that binds directly to heme and regulates lipid and drug metabolism and hormone signaling, was proposed as the complex where resides the σ₂R binding site [Xu, J. et al., Nat. Commun., (2011), 2, 380]. Finally, in 2017, the σ₂R subtype was purified and identified as transmembrane protein-97 (TMEM97), an endoplasmic-reticulum-resident molecule implicated in cholesterol homeostasis due to its association with the lysosomal Niemann-Pick cholesterol transporter type 1 (NPC1) [Alon, A. et al., Proc. Natl. Acad. Sci. USA, (2017), 114, 7160-7165; Ebrahimi-Fakhari, D. et al., Human Molecular Genetics, (2016), 25, 3588-3599]. The role of σ₂ receptor in cholesterol pathways was known since the 1990s and recent studies published by Mach et al. on modulation of trafficking and internalization of LDL by the formation of a ternary complex between LDLR, PGRMC1 and TMEM97, reinforces this association [Moebius, F. F. et al., Trends Pharmacol. Sci., (1997), 18, 67-70; Riad, A. et al., Sci. Rep., (2018), 8, 16845].

σ₂R/TMEM97, previously known also as meningioma-associated protein, MAC30, is expressed in various normal and diseased human tissues and up-regulation in certain tumors and down-regulation in other suggested that this protein played a distinct role in human malignancies. The cloning of σ₂ receptor confirmed its overexpression in epithelial, colorectal, ovarian lung and breast cancers [Moparthi, S. B. et al., Int. J. Oncol., (2007), 30, 91-95; Yan, B. Y. et al., Chemotherapy, (2010), 56, 424-428; Zhao, Z. R.; Chemotherapy, (2011), 57, 394-401; Ding, H. et al., Asian Pac. J. Cancer Prev., (2016), 17, 2705-2710]. σ₂R/TMEM97 has a molecular weight of 18-21.5 kDa and its sequence predicts a four transmembrane domain protein with cytosolic N and C terminal [Hellewell, S. B. et al., Eur. J. Pharmacol. Mol. Pharmacol. Sect., (1994), 268, 9-18]. The potential signal transduction of σ₂ receptor is not yet understood, but it seems to modulate Ca²⁺ and K⁺ channels, and to interact with caspases, epidermal growth factor receptor (EGFR), and with mammalian target of rapamycin, mTOR, signaling pathways [Vilner, B. J. et al., J. Pharmacol. Exp. Ther., (2000), 292, 900-911; Wilke, R. A. et al., J. Biol. Chem., (1999), 274, 18387-18392; Huang, Y.-S. et al., Med. Res. Rev., (2014), 34, 532-566]. These findings would explain the apoptotic effect of some σ₂ ligands by lysosome dysfunction, reactive oxygen species (ROS) production and caspase-dependent events [Ostenfeld, M. S. et al., Autophagy, (2008), 4, 487-499; Hornick, J. R. et al., J. Exp. Clin. Cancer Res., (2012), 31, 41; Zeng, C. et al., Br. J. Cancer, (2012), 106, 693-701; Pati, M. L. et al., BMC Cancer, (2017), 17, 51].

The σ₂ receptor is involved also in dopaminergic transmission, microglia activation, and neuroprotection [Guo, L. et al., Curr. Med. Chem. (2015), 22, 989-1003]. Terada et al. published in 2018 that σ₂ ligands enhance nerve growth factor (NGF)-induced neurite outgrowth in PC12 cells [Terada, K. et al., Plos One, (2018), 13, e0209250]. The σ₂ receptor plays a key role in amyloid β (Aβ)-induced synaptotoxicity, and σ₂ receptor ligands that block the interaction of Aβ oligomers with the σ₂ receptor have been shown to be neuroprotective [Izzo, N. J. et al., Plos One, (2014), 9, e111899]. σ₂ receptor modulators improve cognitive performance in a transgenic mouse model of Alzheimer's disease (AD), and in two mouse traumatic brain injury models, and could also reduce ischemic stroke injury by enhancing glial cell survival, blocking ischemia-induced glial cell activation, and decreasing nitrosative stress [Katnik, C. et al., J. Neurochem., (2016), 139, 497-509; Yi, B. et al., J. Neurochem., (2017), 140, 561-575; Vázquez-Rosa, E. et al., ACS Chem. Neurosci., (2019), 10, 1595-1602]. The σ₂ receptor has been implicated in other neurological disorders as schizophrenia [Harvey, P.D. et al., Schizophrenia Research (2020), 215, 352-356], alcohol abuse [Scott, L. L. et al., Neuropsychopharmacology, (2018), 43, 1867-1875] and pain [Sahn, J. J. et al., ACS Chem. Neurosci., (2017), 8, 1801-1811]. Norbenzomorphan UKH-1114, a σ₂ ligand, relieved mechanical hypersensitivity in the spared nerve injury (SNI) mice model of neuropathic pain, an effect explained by the preferential expression of σ₂R/TMEM97 gene in structures involved in pain such as the dorsal root ganglion (DRG).

The σ₂ receptor requires two acidic groups (Asp29, Asp56) for ligand binding, similar to σ₁R, which requires Asp126 and Glu172. σ₁R and σ₂R might have similarities in their binding sites but not necessarily other structural similarities if their amino acid sequences are compared. As σ₁R, σ₂ receptor interacts with a wide range of signaling proteins, receptors and channels, but the question if σ₂ receptor has a primarily structural or a modulatory activity remains to be answered. Several classes of σ₂ receptor ligands have been developed since Perregaard et al., synthesized Siramesine and indole analogues in 1995 [Perregaard, J. et al., J. Med. Chem., (1995), 38, 1998-2008]: tropanes [Bowen, W. D. et al., Eur. J. Pharmacol., (1995), 278, 257-260], norbenzomorphans [Sahn, J. J. et al., ACS Med. Chem. Lett., (2017), 8, 455-460], tetrahydroisoquinolines [Sun, Y.-T. et al., Eur. J. Med. Chem., (2018), 147, 227-237] or isoindolines [Grundmana, M. et al., Alzheimer's & Dementia: Translational Research & Clinical Interventions, (2019), 5, 20-26] amongst others [Berardi, F. et al., J. Med. Chem., (2004), 47, 2308-2317]. Many of these ligands present a lack of selectivity over serotoninergic receptors but mainly, there is a difficulty to reach a high selectivity over σ_{1.} Several σ₁-selective ligands are available, but ligands with high selectivity for σ₂ over σ₁ are relatively scarce. A significant challenge for the study of the σ₂ receptor is the paucity of highly σ₂-selective ligands.

In view of the potential therapeutic applications of agonists or antagonists of the sigma receptor, a great effort has been directed to find selective ligands. Thus, the prior art has disclosed different sigma receptor ligands, as outlined above.

Nevertheless, there is still a need to find compounds having pharmacological activity towards the sigma receptor, being both effective, selective, and/or having good “drugability” properties, i.e. good pharmaceutical properties related to administration, distribution, metabolism and excretion.

Surprisingly, it has been observed that the new compounds described in the present invention show a selective affinity for sigma receptors. These compounds are therefore particularly suitable as pharmacologically active agents in medicaments for the prophylaxis and/or treatment of disorders or diseases related to sigma receptors.

### SUMMARY OF THE INVENTION

The present invention discloses novel compounds with great affinity to sigma receptors which might be used for the treatment of sigma related disorders or diseases. In particular, the compounds of the invention can be useful for the treatment of pain and pain related disorders and/or CNS (Central Nervous System) disorders.

The invention is directed in a main aspect to a compound of Formula (I′),

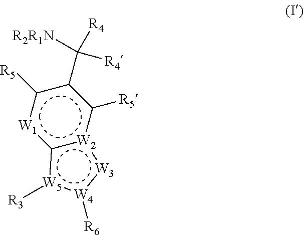

wherein R₁, R₂, R₃, R₄, R_{4′}, R₅, R_{5′}, and R₆ are as defined below in the detailed description.

A further aspect of the invention refers to the processes for preparation of compounds of formula (I′).

Another aspect of the invention refers to a compound of formula (I′) for use as a medicament.

It is also an aspect of the invention a pharmaceutical composition comprising a compound of formula (I′).

Finally, it is an aspect of the invention a compound of formula (I′) for use in therapy and more particularly for the treatment of pain and pain related conditions and/or CNS (Central Nervous System) disorders.

### DETAILED DESCRIPTION OF THE INVENTION

The invention is directed to a family of new compounds, which show pharmacological activity towards the sigma receptors; thus, solving the above problem of identifying alternative or improved pain and/or CNS treatments by offering such compounds.

The applicant has found that the problem of providing a new effective and alternative solution for treating pain and pain related disorders and/or CNS (Central Nervous System) disorders can surprisingly be solved by using compounds binding to the sigma receptors.

In a first aspect, the present invention is directed to a compound of formula (I′):

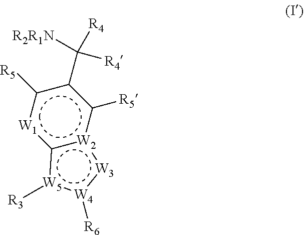

Wherein

W₁, W₂, W₃, W₄ and W₅ represent independently from one another a N atom, a C atom or a —CH— group with the proviso that irrespective of their meaning the rings are always aromatic as represented by the dotted lines

R₁ and R₂ are independently from one another hydrogen or a linear or branched C₁-C₆ alkyl radical or, alternatively, R₁ and R₂ together with the N atom to which they are attached, form a saturated heterocycle optionally containing a further heteroatom selected from N or O and optionally substituted by at least one of a C₁-C₆ alkyl radical, a halogen atom, a C₁-C₆ haloalkyl radical, a —(CH₂)ₙOR group, an aryl group, a 3 to 6 membered saturated heterocycle containing at least one heteroatom selected from N or O, or alternatively R₁ and R₂ together with the N atom to which they are attached, form a saturated spiro-heterocycle containing optionally an additional heteroatom selected from O, N or S and being optionally substituted by at least one of a C₁-C₆ alkyl radical, a halogen atom, a C₁-C₆ haloalkyl radical, a —(CH₂)ₙOR group;

R is a H atom or a C₁-C₆ alkyl radical;

n is 0 or 1

R₃ is a pyridinyl radical optionally substituted by a C₁-C₆ alkyl radical, a halogen atom, a C₁-C₆ haloalkyl radical, a C₁₋₆ alkoxy group, a hydroxy group or a cyano group;

R₄, R₄′ are independently from one another hydrogen or a linear or branched C₁-C₆ alkyl radical;

R₅ and R₅′ are independently from one another hydrogen, halogen, C₁₋₆-alkyl, C₁₋₆-haloalkyl, C₁₋₆ alkoxy, —CN;

R₆ is hydrogen, halogen, C₁₋₆-alkyl, C₁₋₆-haloalkyl, C₁₋₆ alkoxy, —CN;

wherein the compound of formula (I′) is optionally in form of one of the stereoisomers, preferably enantiomers or diastereomers, of a racemate or in form of a mixture of at least two of the stereoisomers, preferably enantiomers and/or diastereomers, in any mixing ratio, or a corresponding salt, co-crystal, solvate or prodrug thereof.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds of general formula (I′) or their salts, cocrystals or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I′), or of its salts, solvates or prodrugs.

For the sake of clarity the expression “a compound according to formula (I′), wherein R₁, R₂, R₃, R₄, R_{4′}, R₅, R_{5′}, and R₆ are as defined below in the detailed description” would (just like the expression “a compound of formula (I′) as defined in the claims) refer to “a compound according to formula (I′)”, wherein the definitions of the respective substituents R₁ etc. (also from the cited claims) are applied.

For clarity purposes, all groups and definitions described in the present description and referring to compounds of formula (I′), also apply to all intermediates of synthesis.

“Halogen” or “halo” as referred in the present invention represent fluorine, chlorine, bromine or iodine. When the term “halo” is combined with other substituents, such as for instance “C₁₋₆ haloalkyl” or “C₁₋₆ haloalkoxy” it means that the alkyl or alkoxy radical can respectively contain at least one halogen atom.

“C₁₋₆ alkyl”, as referred to in the present invention, are saturated aliphatic radicals. They may be unbranched (linear) or branched and are optionally substituted. C₁₋₆-alkyl as expressed in the present invention means an alkyl radical of 1, 2, 3, 4, 5 or 6 carbon atoms. Preferred alkyl radicals according to the present invention include but are not restricted to methyl, ethyl, propyl, n-propyl, isopropyl, butyl, n-butyl, tert-butyl, isobutyl, sec-butyl, 1-methylpropyl, 2-methylpropyl, 1,1-dimethylethyl, pentyl, n-pentyl, 1,1-dimethylpropyl, 1,2-dimethylpropyl, 2,2-dimethylpropyl, hexyl, 1-methylpentyl. The most preferred alkyl radicals are C₁₋₄ alkyl, such as methyl, ethyl, propyl, n-propyl, isopropyl, butyl, n-butyl, tert-butyl, isobutyl, sec-butyl, 1-methylpropyl, 2-methylpropyl or 1,1-dimethylethyl. Alkyl radicals, as defined in the present invention, are optionally mono- or polysubstituted by substitutents independently selected from a halogen, branched or unbranched C₁₋₆-alkoxy, branched or unbranched C₁₋₆-alkyl, C₁₋₆-haloalcoxy, C₁₋₆-haloalkyl, trihaloalkyl or a hydroxyl group.

“C₁₋₆ alkoxy” as referered to in the present invention, is understood as meaning an alkyl radical as defined above attached via oxygen linkage to the rest of the molecule. Examples of alkoxy include, but are not limited to methoxy, ethoxy, propoxy, butoxy or tert-butoxy.

A heterocyclyl radical (Het) or group (also called heterocyclyl hereinafter) is understood as meaning 3 to 9 membered mono or fused polycyclic heterocyclic ring systems, with at least one saturated ring which contains one or more heteroatoms selected from the group consisting of nitrogen, oxygen and/or sulfur in the ring. A heterocyclic group can also be substituted once or several times.

Subgroups inside the heterocyclyls as understood herein include heteroaryls and non-aromatic heterocyclyls.
the heteroaryl (being equivalent to heteroaromatic radicals or aromatic heterocyclyls) is an aromatic 3 to 9 membered mono or fused polycyclic heterocyclic ring system of one or more rings of which at least one aromatic ring contains one or more heteroatoms from the group consisting of nitrogen, oxygen and/or sulfur in the ring; preferably it is a 3 to 9 membered mono or fused polycyclic aromatic heterocyclic ring system of one or two rings of which at least one aromatic ring contains one or more heteroatoms selected from the group consisting of nitrogen, oxygen and/or sulfur in the ring; more preferably it is selected from furan, benzofuran, thiophene, benzothiophene, pyrrole, pyridine, pyrimidine, pyrazine, benzothiazole, indole, benzotriazole, thiazole, imidazole, pyrazole, oxazole, oxadiazoleand benzimidazole;
the non-aromatic heterocyclyl is a 3 to 9 membered mono or fused polycyclic heterocyclic ring system of one or more rings of which at least one ring—with this (or these) ring(s) then not being aromatic—contains one or more heteroatoms from the group consisting of nitrogen, oxygen and/or sulfur in the ring; preferably it is a 3 to 9 membered mono or fused polycyclic heterocyclic ring system of one or two rings of which one or both rings—with this one or two rings then not being aromatic—contain/s one or more heteroatoms selected from the group consisting of nitrogen, oxygen and/or sulfur in the ring, more preferably it is selected from azetidine, oxetane, tetrahydrofuran, oxazepane, pyrrolidine, piperidine, piperazine, tetrahydropyran, morpholine, indoline, oxopyrrolidine, especially is piperazine, morpholine, tetrahydropyran, piperidine, oxopyrrolidine and pyrrolidine.

Preferably, in the context of this invention heterocyclyl is defined as a 3 to 9 membered mono or fused polycyclic ring system of one or more saturated rings of which at least one ring contains one or more heteroatoms selected from the group consisting of nitrogen, oxygen and/or sulfur in the ring. Preferably it is a 3 to 9 membered mono or fused polycyclic heterocyclic ring system of one or two saturated rings of which at least one ring contains one or more heteroatoms selected from the group consisting of nitrogen, oxygen, and sulfur in the ring. More preferably, it is a 3 to 6 membered mono or bicyclic heterocyclyl ring system containing one nitrogen atom and optionally a second heteroatom selected from nitrogen and oxygen. In another preferred embodiment of the invention, said heterocyclyl is a substituted mono or bicyclic heterocyclyl ring system.

Preferred examples of saturated heterocyclyls include azetidine, azepane, oxetane, tetrahydrofuran, oxazepane, pyrrolidine, piperidine, piperazine, tetrahydropyran, morpholine, 2,7-diazaspiro[3.5]nonane, 2,7-diazaspiro[4.4]nonane, octahydropyrrolo[3,4-c]pyrrole.

An N-containing heterocyclyl is a heterocyclic ring system of one or more saturated rings of which at least one ring contains a nitrogen and optionally one or more further heteroatoms selected from the group consisting of nitrogen, oxygen and/or sulfur in the ring; preferably is a heterocyclic ring system of one or two saturated rings of which at least one ring contains a nitrogen and optionally one or more further heteroatoms selected from the group consisting of nitrogen, oxygen and/or sulfur in the ring, more preferably is selected from azetidine, azepane, oxazepam, pyrrolidine, piperazine, 2,7-diazaspiro[3.5]nonane, 2,7-diazaspiro[4.4]nonane or octahydropyrrolo[3,4-c]pyrrole.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

“Heterocycloalkyl” as referred to in the present invention, are understood as meaning saturated, generally 5 or 6 membered cyclic hydrocarbons which can optionally be unsubstituted, mono- or polysubstituted and which have at least one heteroatom in their structure selected from N, O or S. Examples for heterocycloalkyl radical preferably include but are not restricted to pyrrolidine, aziridine, azetidine, tetrahydropyrrole, oxirane, oxetane, dioxetane, tetrahydropyrane, tetrahydrofurane, dioxane, dioxolane, oxazolidine, piperidine, piperazine, morpholine, azepane or diazepane. Heterocycloalkyl radicals, as defined in the present invention, are optionally mono- or polysubstituted by substitutents independently selected from a halogen atom, branched or unbranched C₁₋₆-alkyl, branched or unbranched C₁₋₆-alkoxy, C₁₋₆-haloalkoxy, C₁₋₆-haloalkyl, trihaloalkyl or a hydroxyl group. More preferably heterocycloalkyl in the context of the present invention are 5 or 6-membered ring systems optionally at least monosubstituted.

A heterocycloalkylalkyl group/radical C_{1-6,} as defined in the present invention, comprises a linear or branched, optionally at least mono-substituted alkyl chain of 1 to 6 atoms which is bonded to a cycloalklyl group, as defined above. The heterocycloalkylalkyl radical is bonded to the molecule through the alkyl chain. A preferred heterocycloalkylalkyl group/radical is a piperidinethyl group or a piperazinylmethyl group, wherein the alkyl chain is optionally branched or substituted. Preferred substituents for cycloalkylalkyl group/radical, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

“Aryl” as referred to in the present invention, is understood as meaning ring systems with at least one aromatic ring but without heteroatoms even in only one of the rings. These aryl radicals may optionally be mono-or polysubstituted by substitutents independently selected from a halogen atom, —CN, branched or unbranched C₁₋₆-alkyl, branched or unbranched C₁₋₆-alkoxy, C₁₋₆-haloalcoxy, C₁₋₆-haloalkyl, a heterocyclyl group and a hydroxyl group. Preferred examples of aryl radicals include but are not restricted to phenyl, naphthyl, fluoranthenyl, fluorenyl, tetralinyl, indanyl or anthracenyl radicals, which may optionally be mono- or polysubstituted, if not defined otherwise. More preferably aryl in the context of the present invention is a 6-membered ring system optionally at least mono or polysubstituted.

An arylalkyl radical C1-6, as defined in the present invention, comprises an unbranched or branched, optionally at least mono-substituted alkyl chain of 1 to 6 carbon atoms which is bonded to an aryl group, as defined above. The arylalkyl radical is bonded to the molecule through the alkyl chain. A preferred arylalkyl radical is a benzyl group or a phenetyl group, wherein the alkyl chain is optionally branched or substituted. Preferred substituents for arylalkyl radicals, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

Heteroarylalkyl group/radical C₁₋₆ as defined in the present invention, comprises a linear or branched, optionally at least mono-substituted alkyl chain of 1 to 6 carbon atoms which is bonded to an heteroaryl group, as defined above. The heteroarylalkyl radical is bonded to the molecule through the alkyl chain. Preferred substituents for heteroarylalkyl radicals, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

The term “condensed” according to the present invention means that a ring or ring-system is attached to another ring or ring-system, whereby the terms “annulated” or “annelated” are also used by those skilled in the art to designate this kind of attachment.

The term “ring system” according to the present invention refers to a system consisting of at least one ring of connected atoms but including also systems in which two or more rings of connected atoms are joined with “joined” meaning that the respective rings are sharing one (like a spiro structure), two or more atoms being a member or members of both joined rings. The “ring system” thus defined comprises saturated, unsaturated or aromatic carbocyclic rings which contain optionally at least one heteroatom as ring member and which are optionally at least mono-substituted and may be joined to other carbocyclic ring systems such as aryl radicals, heteroaryl radicals, cycloalkyl radicals etc.

The terms “condensed”, “annulated” or “annelated” are also used by those skilled in the art to designate this kind of join.

A leaving group (LG) is a group that in a heterolytic bond cleavage keeps the electron pair of the bond. Suitable leaving groups are well known in the art and include CI, Br, I and —O—SO₂R¹⁴, wherein R¹⁴ is F, C₁₋₄-alkyl, C₁₋₄-haloalkyl, or optionally substituted phenyl. The preferred leaving groups are Cl, Br, I, tosylate, mesylate, triflate, nonaflate and fluorosulphonate.

“Protecting group” is a group that is chemically introduced into a molecule to avoid that a certain functional group from that molecule undesirably reacts in a subsequent reaction. Protecting groups are used, among others, to obtain chemoselectivity in chemical reactions. The preferred protecting group in the context of the invention are Boc (tert-butoxycarbonyl) or Teoc (2-(trimethylsilyl)ethoxycarbonyl).

The term “salt” is to be understood as meaning any form of the active compound according to the invention in which this assumes an ionic form or is charged and is coupled with a counter-ion (a cation or anion). The definition particularly includes physiologically acceptable salts, this term must be understood as equivalent to “pharmaceutically acceptable salts”.

The term “pharmaceutically acceptable salts” in the context of this invention means any salt that is tolerated physiologically (normally meaning that it is not toxic, particularly as a result of the counter-ion) when used in an appropriate manner for a treatment, particularly applied or used in humans and/or mammals. This definition specifically includes in the context of this invention a salt formed by a physiologically tolerated acid, i.e. salts of a specific active compound with physiologically tolerated organic or inorganic acids—particularly when used on humans and/or mammals. Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid. In addition, the pharmaceutically acceptable salts may be formed with a physiologically tolerated cation, preferably inorganic, particularly when used on humans and/or mammals. Salts with alkali and alkali earth metals are particularly preferred, as well as those formed with ammonium cations (NH₄⁺). Preferred salts are those formed with (mono) or (di)sodium, (mono) or (di)potassium, magnesium or calcium. These physiologically acceptable salts may also be formed with anions or acids and, in the context of this invention, are understood as being salts formed by at least one compound used in accordance with the invention—normally protonated, for example in nitrogen—such as a cation and at least one physiologically tolerated anion, particularly when used on humans and/or mammals.

The compounds of the invention may be present in crystalline form or in amorphous form.

Any compound that is a solvate of a compound according to formula (I′) defined above is understood to be also covered by the scope of the invention. Methods of solvation are generally known within the art. Suitable solvates are pharmaceutically acceptable solvates. The term “solvate” is to be understood as meaning any form of the active compound according to the invention in which this compound has attached to it via non-covalent binding another molecule (most likely a polar solvent) especially including hydrates and alcoholates, like methanolate or ethanolate.

The term “co-crystal” is to be understood as a crystalline material comprising a specific active compound with at least one additional component, usually a co-crystal former, and of which at least two of the constituents are held together by weak interactions. Weak interaction is being defined as an interaction which is neither ionic nor covalent and includes for example: hydrogen bonds, van der Waals forces, and π-π interactions.

The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the compounds of the invention: esters, amino acid esters, phosphate esters, metal salts sulfonate esters, carbamates, and amides. Examples of well-known methods of producing a prodrug of a given acting compound are known to those skilled in the art and can be found e.g. in Krogsgaard-Larsen et al. “Textbook of Drug design and Discovery” Taylor & Francis (april 2002).

Any compound that is a prodrug of a compound of general formula (I′) is within the scope of the invention. Particularly favored prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Any compound that is an N-oxide of a compound according to the invention like a compound according to formula (I′) defined above is understood to be also covered by the scope of the invention.

The compounds of formula (I′) as well as their salts or solvates are preferably in pharmaceutically acceptable or substantially pure form. By pharmaceutically acceptable pure form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

Unless otherwise defined, all the groups above mentioned that can be substituted or unsubstituted may be substituted at one or more available positions by one or more suitable groups such as a halogen, preferably Cl or F; OR′, ═O, SR′, SOR′, SO₂R′, OSO₂R′, OSO₃R′, NO₂, NHR′, NR′R″, ═N—R′, N(R′)COR′, N(COR′)₂, N(R′)SO₂R′, N(R′)C(═NR′)N(R′)R′, N₃, CN, halogen, COR′, COOR′, OCOR′, OCOOR′, OCONHR′, OCONR′R″, CONHR′, CONR′R″, CON(R′)OR′, CON(R′)SO₂R′, PO(OR′)₂, PO(OR′)R′, PO(OR′)(N(R′)R′), C₁₋₆ alkyl, C₃₋₁₀ cycloalkyl, C₂₋₆ alkenyl, C₂₋₆ alkynyl, aryl, and heterocyclic group, wherein each of the R′ and R″ groups is independently selected from the group consisting of hydrogen, C₁₋₆ alkyl, C₃₋₁₀ cycloalkyl, C₂₋₆ alkenyl, C₂₋₆ alkynyl, aryl and heterocyclic group. Where such groups are themselves substituted, the substituents may be chosen from the foregoing list.

In a particular and preferred embodiment of the invention, R₁ and R₂ are independently from one another, C₁-C₆ alkyl radical, preferably ethyl, propyl or isopropyl; or

R₁ and R₂ together with the N atom to which they are attached form a pyrrolidine ring; a morpholine ring; an oxazepane ring; a piperazine ring optionally substituted by an oxetanyl; or piperidine ring radical, optionally substituted with a group selected from a phenyl radical, a halogen atom and a —(CH₂) OR group; or

R₁ and R₂ together with the N atom form an oxa-azaspiro alkane, preferably oxa-azaspiro-nonane, oxa-azaspiro-decane or oxa-azaspiro-undecane.

In another preferred embodiment R₃ is a pyridinyl radical optionally substituted with halogen, C₁₋₆-alkyl, C₁₋₆-haloalkyl or C₁₆ alkoxy.

In another preferred embodiment, R₄, R₄′, R₅, R₅′ and R₆ is H.

In a particular and preferred embodiment of the invention the compounds of formula (I′) are represented by one of the following subformulas (I), (I′a), (I′b) or (I′c):

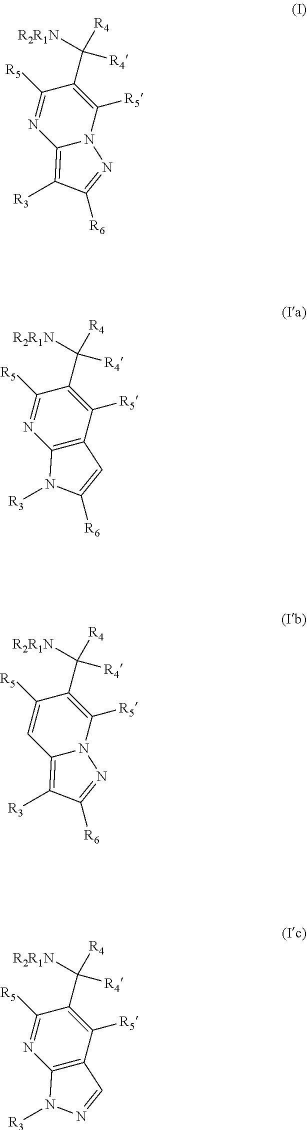

wherein R₁, R₂, R₃, R₄, R₄′, R₅, R₅′ and R₆ are as defined along the description and claims. The most preferred compounds of the invention are those of general formula (I):

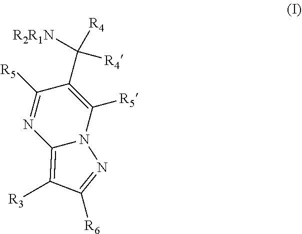

wherein

R₁ and R₂ are independently from one another hydrogen or a linear or branched C₁-C₆ alkyl radical or, alternatively, R₁ and R₂ together with the N atom to which they are attached, form a saturated heterocycle optionally containing a further heteroatom selected from N or O and optionally substituted by at least one of a C₁-C₆ alkyl radical, a halogen atom, a C₁-C₆ haloalkyl radical, an —(CH₂)ₙOR group, an aryl group, a 3 to 6 membered saturated heterocycle containing at least one heteroatom selected from N or O, or alternatively R₁ and R₂ together with the N atom to which they are attached, form a saturated spiro-heterocycle containing optionally an additional heteroatom selected from O, N or S and being optionally substituted by at least one of a C₁-C₆ alkyl radical, a halogen atom, a C₁-C₆ haloalkyl radical, an —(CH2),OR group;

R is a H atom or a C₁-C₆ alkyl radical;

n is 0 or 1

R₃ is a pyridinyl radical optionally substituted by a C₁-C₆ alkyl radical, a halogen atom, a C₁-C₆ haloalkyl radical, a OH group, a C₁₋₆ alkoxy group or a cyano group;

R₄, R₄′ are independently from one another hydrogen or a linear or branched C₁-C₆ alkyl radical;

R₅ and R₅′ are independently from one another hydrogen, halogen, C₁₋₆-alkyl, C₁₋₆-haloalkyl, C₁₋₆ alkoxy, —CN;

R₆ is hydrogen, halogen, C₁₋₆-alkyl, C₁₋₆-haloalkyl, C₁₋₆ alkoxy, —CN; wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof.

The preferred compounds of the invention are selected from:
[1] 6-(Pyrrolidin-1-ylmethyl)-3-(2-(trifluoromethyl)pyridin-4-yl)pyrazolo[1,5-a]pyrimidine;
[2] 3-(Pyridin-4-yl)-6-(pyrrolidin-1-ylmethyl)pyrazolo[1,5-a]pyrimidine;
[3] 3-(3-Fluoropyridin-4-yl)-6-(pyrrolidin-1-ylmethyl)pyrazolo[1,5-a]pyrimidine;
[4] 3-(2-Methoxypyridin-3-yl)-6-(pyrrolidin-1-ylmethyl)pyrazolo[1,5-a]pyrimidine;
[5] 6-((4-Phenylpiperidin-1-yl)methyl)-3-(pyridin-4-yl)pyrazolo[1,5-a]pyrimidine;
[6] 6-((4-Phenylpiperidin-1-yl)methyl)-3-(2-(trifluoromethyl)pyridin-4 yl)pyrazolo[1,5-a]pyrimidine;
[7] 7-((3-(2-(Trifluoromethyl)pyridin-4-yl)pyrazolo[1,5-a]pyrimidin-6-yl)methyl)-1-oxa-7-azaspiro[3.5]nonane;
[8] 7-((3-(3-Fluoropyridin-4-yl)pyrazolo[1,5-a]pyrimidin-6-yl)methyl)-1-oxa-7-azaspiro[3.5]nonane;
[9] 7-((3-(2-(Trifluoromethyl)pyridin-4-yl)pyrazolo[1,5-a]pyrimidin-6-yl)methyl)-2-oxa-7-azaspiro[3.5]nonane;
[10] 8-((3-(2-(Trifluoromethyl)pyridin-4-yl)pyrazolo[1,5-a]pyrimidin-6-yl)methyl)-2-oxa-8-azaspiro[4.5]decane;
[11] 6-((4,4-Difluoropiperidin-1-yl)methyl)-3-(2-(trifluoromethyl)pyridin-4-yl)pyrazolo[1,5-a]pyrimidine;
N-Ethyl-N-((3-(2-(trifluoromethyl)pyridin-4-yl)pyrazolo[1,5-a]pyrimidin-6-yl) methyl)ethanamine;
[13] 7-((3-(6-(Trifluoromethyl)pyridin-3-yl)pyrazolo[1,5-a]pyrimidin-6-yl)methyl)-2-oxa-7-azaspiro[3.5]nonane;
[14] 7-((3-(6-(Trifluoromethyl)pyridin-2-yl)pyrazolo[1,5-a]pyrimidin-6-yl)methyl)-2-oxa-7-azaspiro[3.5]nonane;
[15] 7-((3-(2-Methylpyridin-4-yl)pyrazolo[1,5-a]pyrimidin-6-yl)methyl)-2-oxa-7-azaspiro[3.5]nonane;
[16] 7-((3-(2-Chloropyridin-4-yl)pyrazolo[1,5-a]pyrimidin-6-yl)methyl)-2-oxa-7-azaspiro[3.5]nonane;
[17] 7-((3-(4-(Trifluoromethyl)pyridin-3-yl)pyrazolo[1,5-a]pyrimidin-6-yl)methyl)-2-oxa-7-azaspiro[3.5]nonane
[18] 7-((3-(Pyridin-2-yl)pyrazolo[1,5-a]pyrimidin-6-yl)methyl)-2-oxa-7-azaspiro[3.5]nonane;
[19] 7-((3-(3-(Trifluoromethyl)pyridin-4-yl)pyrazolo[1,5-a]pyrimidin-6-yl)methyl)-2-oxa-7-azaspiro[3.5]nonane;
[20] 7-((2-Methyl-3-(2-(trifluoromethyl)pyridin-4-yl)pyrazolo[1,5-a]pyrimidin-6-yl) methyl)-2-oxa-7-azaspiro[3.5]nonane;
[21] 6-((4-Methoxypiperidin-1-yl)methyl)-3-(6-(trifluoromethyl)pyridin-3-yl) pyrazolo[1,5-a]pyrimidine;
[22] 2-((3-(6-(Trifluoromethyl)pyridin-3-yl)pyrazolo[1,5-a]pyrimidin-6-yl)methyl)-7-oxa-2-azaspiro[3.5]nonane;
[23] 6-((4-Methoxypiperidin-1-yl)methyl)-3-(2-methylpyridin-4-yl)pyrazolo[1,5-a]pyrimidine;
[24] 4-((3-(2-Methylpyridin-4-yl)pyrazolo[1,5-a]pyrimidin-6-yl)methyl)-1,4-oxazepane;
[25] 6-((4-(Methoxymethyl)piperidin-1-yl)methyl)-3-(2-methylpyridin-4-yl) pyrazolo[1,5-a]pyrimidine;
[26] 9-((3-(2-Methylpyridin-4-yl)pyrazolo[1,5-a]pyrimidin-6-yl)methyl)-3-oxa-9-azaspiro[5.5]undecane;
[27] 2-((3-(2-Methylpyridin-4-yl)pyrazolo[1,5-a]pyrimidin-6-yl)methyl)-7-oxa-2-azaspiro[3.5]nonane;
[28] 6-((4-(Methoxymethyl)piperidin-1-yl)methyl)-3-(2-(trifluoromethyl)pyridin-4-yl) pyrazolo[1,5-a]pyrimidine;
[29] 4-((3-(2-(Trifluoromethyl)pyridin-4-yl)pyrazolo[1,5-a]pyrimidin-6-yl) methyl)morpholine;
[30] 6-((4-(Ethoxymethyl)piperidin-1-yl)methyl)-3-(2-(trifluoromethyl)pyridin-4-yl) pyrazolo[1,5-a]pyrimidine;
[31] 4-((3-(2-(Trifluoromethyl)pyridin-4-yl)pyrazolo[1,5-a]pyrimidin-6-yl)methyl)-1,4-oxazepane;
[32] 9-((3-(2-(Trifluoromethyl)pyridin-4-yl)pyrazolo[1,5-a]pyrimidin-6-yl)methyl)-3-oxa-9-azaspiro[5.5]undecane;
[33] 2-((3-(2-(Trifluoromethyl)pyridin-4-yl)pyrazolo[1,5-a]pyrimidin-6-yl)methyl)-7-oxa-2-azaspiro[3.5]nonane;
[34] 6-((4-Methoxypiperidin-1-yl)methyl)-3-(2-(trifluoromethyl)pyridin-4-yl) pyrazolo[1,5-a]pyrimidine;
[35] 6-((4-(Oxetan-3-yl)piperazin-1-yl)methyl)-3-(2-(trifluoromethyl)pyridin-4-yl) pyrazolo[1,5-a]pyrimidine;
[36] 1-((3-(2-(Trifluoromethyl)pyridin-4-yl)pyrazolo[1,5-a]pyrimidin-6-yl) methyl)piperidin-4-ol;
[37] 7-((5,7-Dimethyl-3-(2-(trifluoromethyl)pyridin-4-yl)pyrazolo[1,5-a]pyrimidin-6-yl) methyl)-2-oxa-7-azaspiro[3.5]nonane;
[38] 7-((1-(2-(Trifluoromethyl)pyridin-4-yl)-1H-pyrrolo[2,3-b]pyridin-5-yl)methyl)-2-oxa-7-azaspiro[3.5]nonane;
[39] 5-((4-(Methoxymethyl)piperidin-1-yl)methyl)-1-(2-(trifluoromethyl)pyridin-4-yl)-1H-pyrrolo[2,3-b]pyridine;
[40] 5-((4-(Ethoxymethyl)piperidin-1-yl)methyl)-1-(2-(trifluoromethyl)pyridin-4-yl)-1H-pyrrolo[2,3-b]pyridine;
[41] 5-((4-(Methoxymethyl)piperidin-1-yl)methyl)-1-(6-(trifluoromethyl)pyridin-3-yl)-1H-pyrrolo[2,3-b]pyridine;
[42] 7-((1-(6-(Trifluoromethyl)pyridin-3-yl)-1H-pyrrolo[2,3-b]pyridin-5-yl)methyl)-2-oxa-7-azaspiro[3.5]nonane;
[43] 8-((1-(2-(Trifluoromethyl)pyridin-4-yl)-1H-pyrrolo[2,3-b]pyridin-5-yl)methyl)-2-oxa-8-azaspiro[4.5]decane;
[44] 5-((4-(Methoxymethyl)piperidin-1-yl)methyl)-1-(2-(trifluoromethyl)pyridin-3-yl)-1H-pyrrolo[2,3-b]pyridine;
[45] 9-((1-(2-(Trifluoromethyl)pyridin-3-yl)-1H-pyrrolo[2,3-b]pyridin-5-yl)methyl)-3-oxa-9-azaspiro[5.5]undecane;
[46] 9-((1-(2-(Trifluoromethyl)pyridin-4-yl)-1H-pyrrolo[2,3-b]pyridin-5-yl)methyl)-3-oxa-9-azaspiro[5.5]undecane;
[47] 2-((1-(2-(Trifluoromethyl)21yridine-4-yl)-1H-pyrrolo[2,3-b]21yridine-5-yl) methyl)-7-oxa-2-azaspiro[3.5]nonane;
[48] 9-((1-(6-(Trifluoromethyl)pyridin-3-yl)-1H-pyrrolo[2,3-b]pyridin-5-yl)methyl)-3-oxa-9-azaspiro[5.5]undecane;
[49] 5-((4-Methoxypiperidin-1-yl)methyl)-1-(2-(trifluoromethyl)pyridin-4-yl)-1H-pyrrolo[2,3-b]pyridine;
[50] 4-((1-(2-(Trifluoromethyl)pyridin-4-yl)-1H-pyrrolo[2,3-b]pyridin-5-yl)methyl)-1,4-oxazepane;
[51] 5-((4-Methoxypiperidin-1-yl)methyl)-1-(6-(trifluoromethyl)pyridin-3-yl)-1H-pyrrolo[2,3-b]pyridine;
[52] 4-((1-(6-(Trifluoromethyl)pyridin-3-yl)-1H-pyrrolo[2,3-b]pyridin-5-yl)methyl)-1,4-oxazepane;
[53] 3-(5-((3-Oxa-9-azaspiro[5.5]undecan-9-yl)methyl)-1H-pyrrolo[2,3-b]pyridin-1-yl) picolinonitrile;
[54] 8-((1-(2-(Trifluoromethyl)pyridin-3-yl)-1H-pyrrolo[2,3-b]pyridin-5-yl)methyl)-2-oxa-8-azaspiro[4.5]decane;
[55] 2-((1-(6-(Trifluoromethyl)pyridin-3-yl)-1H-pyrrolo[2,3-b]pyridin-5-yl)methyl)-7-oxa-2-azaspiro[3.5]nonane;
[56] 2-((1-(2-(Trifluoromethyl)pyridin-3-yl)-1H-pyrrolo[2,3-b]pyridin-5-yl)methyl)-7-oxa-2-azaspiro[3.5]nonane;
[57] 7-((3-(2-(Trifluoromethyl)pyridin-4-yl)pyrazolo[1,5-a]pyridin-6-yl)methyl)-2-oxa-7-azaspiro[3.5]nonane;
[58] 7-((1-(2-(Trifluoromethyl)pyridin-4-yl)-1H-pyrazolo[3,4-b]pyridin-5-yl) methyl)-2-oxa-7-azaspiro[3.5]nonane;
[59] (1-((3-(2-(Trifluoromethyl)pyridin-4-yl)pyrazolo[1,5-a]pyrimidin-6-yl) methyl)piperidine-4,4-diyl)dimethanol and
[60] (4-(Chloromethyl)-1-((3-(2-(trifluoromethyl)pyridin-4-yl)pyrazolo[1,5-a]pyrimidin-6-yl) methyl)piperidin-4-yl)methanol.

In another aspect, the invention refers to the processes for obtaining compounds of general formula (I). Several procedures have been developed for obtaining all the compounds of the invention, and the procedures will be explained below in methods A and B.

The obtained reaction products may, if desired, be purified by conventional methods, such as crystallization and chromatography. Where the processes described below for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

### Method A

Method A represents a first process for synthetizing compounds according to general formula (I).

Thus, a process is described for the preparation of a compound of general formula (I):

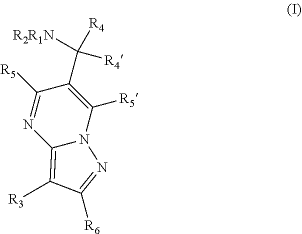

comprising the reaction of a compound of formula (VI):

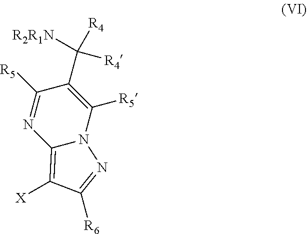

with a compound of formula (VII):

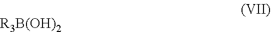

wherein R₁, R₂, R₃, R₄, R₄′, R₅, R₅′, R₆ are as defined along the detailed description and X is a halogen atom.

The compounds of formula (I) may be prepared by the process described in Scheme 1:

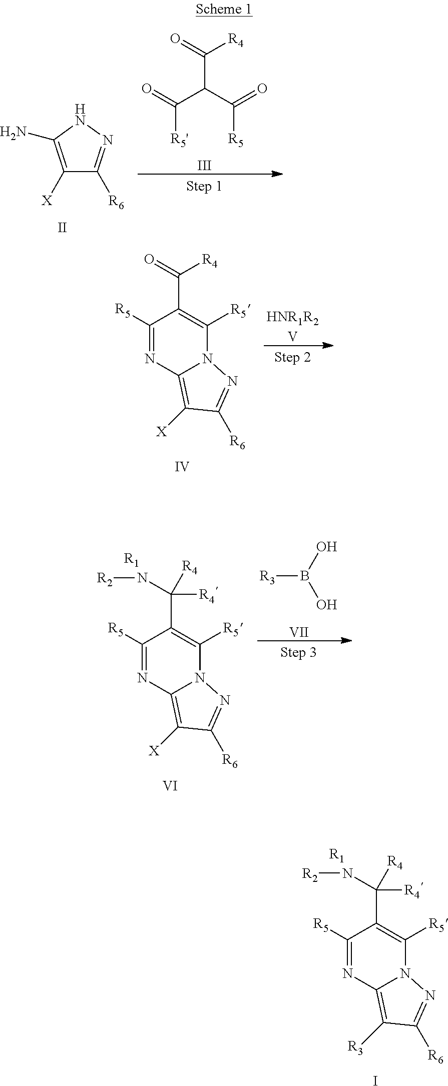

wherein R₁, R₂, R₃, R₄, R_{4′}, R₅, R_{5′} and R₆ have the meanings as defined in claims and/or along the description and X represents a halogen atom.

The process can be carried out as described below:

Step 1: A compound of formula (IV) can be prepared by treating an aminopyrazole of formula (II) with a tricarbonyl compound of formula (III) in a suitable solvent, such as ethanol, at a suitable temperature, preferably heating; followed by treatment with a strong acid, such as trifluoroacetic acid, in a suitable solvent, such as dichloromethane, at a suitable temperature, preferably room temperature.

Step 2: A compound of formula (VI) can be prepared by treating a compound of formula (IV) with an amine of formula (V) in the presence of a suitable reducing reagent, such as sodium triacetoxyborohydride, in a suitable solvent, such as dichloromethane, at a suitable temperature, preferably room temperature.

Step 3: A compound of formula (I) can be prepared by treating a compound of formula (VI) with a boronic acid of formula (VII) (or alternatively with the corresponding boronic ester) in the presence of a Pd catalyst, such as Pd(PPh₃)₄ or Pd(dppf)₂Cl₂ and a suitable base, such as potassium carbonate, in a suitable solvent, such as a mixture of DME/water (1:1) or dioxane, at a suitable temperature, preferably heating, optionally under microwave irradiation.

In addition, a compound of formula (I) can be obtained in enantiopure form by resolution of a mixture of enantiomers or diastereomers of formula (I) either by chiral preparative HPLC or by crystallization of a diastereomeric salt or co-crystal. Alternatively, the resolution step can be carried out at a previous stage, using any suitable intermediate.

The compounds of formula (II), (III), (V) and (VII) used in the methods disclosed above are commercially available or can be synthesized following common procedures described in the literature and exemplified in the synthesis of some intermediates.

### Method B

Alternative processes for the preparation of compounds of formula (Ia) are described in Scheme 2.

Thus, a process is described for the preparation of a compound of general formula (Ia):

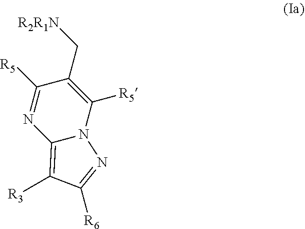

comprising the reaction of a compound of formula (XII):

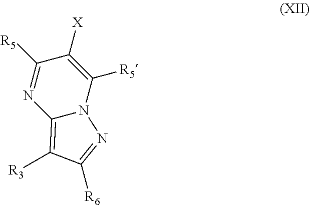

with a compound of formula (XIII):

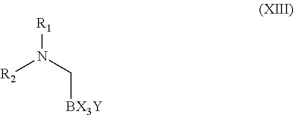

wherein R₁, R₂, R₃, R₅, R₅′, R₆ are as defined in the claims and/or along the description and X is a halogen and Y is an alkaline atom.

The compounds of formula (Ia) may be prepared by the process described in Scheme 2:

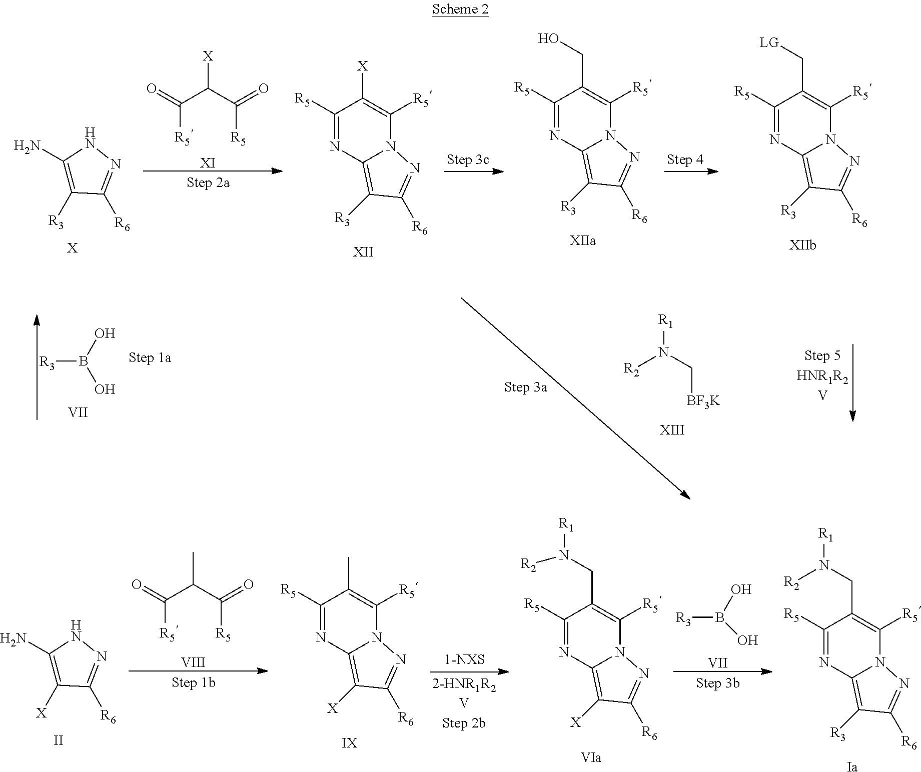

The process involves the Suzuki reaction of an aminopyrazole of formula (II) with a boronic acid of formula (VII) in the conditions described above for Step 3 in Method A, to provide a pyrazole of formula (X) (Step 1a), followed by cyclization with a compound of formula (XI) in the conditions described above for Step 1 in Method A to provide a compound of formula (XII) (Step 2a). A compound of formula (la) is obtained by Molander reaction of a compound of formula (XII) with a compound of formula (XIII) by coupling using a Pd catalyst such as palladium acetate, using a suitable base such as cesium carbonate, and a suitable phosphine, such as XPhos, in a suitable solvent, such as mixtures of dioxane:water , at a suitable temperature, preferably heating and optionally under microwave irradiation (Step 3a). A compound of formula (XIII) may be obtained by reaction of a compound of formula (V) with potassium (bromomethyl)trifluoroborate, using a suitable base such as triethylamine, in a suitable solvent, such as mixtures of tetrahydrofuran/tert-butanol, at a suitable temperature, preferably heating. Alternatively, a compound of formula (Ia) is obtained using a three-step sequence (step 3c, 4 and 5). Hence, by coupling a compound of formula (XII) with potassium (acetoxymethyl)trifluoroborate using a Pd catalyst such as palladium dibenzylideneacetone, a suitable base such as sodium carbonate, and a suitable phosphine, such as RuPhos in a suitable solvent such as mixtures of dioxane:water, at a suitable temperature, preferably heating and optionally under microwave irradiation provides intermediate (XIIa) (Step 3c). This is followed by activation of the alcohol function either via mesylate or bromine substitution using a suitable reagent such as mesyl chloride or phosphorus tribromide, optionally in the presence of an organic base such as triethylamine or diisopropylethylamine, in a suitable solvent, such as dichloromethane or acetonitrile, at a suitable temperature, preferably room temperature to provide intermediate (XIIb) (step 4) and reaction with an amine of formula (V) using a suitable organic base such as triethylamine or diisopropylethylamine, in a suitable solvent, such as acetonitrile, at a suitable temperature, preferably room temperature (Step 5). Steps 4 and 5 can also be performed in one step without isolating the activated alcohol intermediate.

The preparation of pyrazoles of formula (X) can also be accomplished by an alternative process to that described in Scheme 2, such as the reaction of a bromoderivative of formula R₃X with acetonitrile and buthyl lithium in a suitable solvent, such as tetrahydrofuran, at a suitable temperature, preferably between −78° C. and −30° C.; followed by enamine formation with dimethyl formamide dimethyl acetal in a suitable solvent, such as toluene, at a suitable temperature, preferably heating and followed by pyrazole formation with hydrazine hydrate in a suitable solvent, such as ethanol, at a suitable temperature, preferably heating.

A further alternative process involves the preparation of a compound of formula (VI) where R₄ and R_{4′} are hydrogen (VIa) by reacting a compound of formula (II) with a dicarbonyl derivative of formula (VIII) with the conditions described above for Step 1 in Method A (Step 1b), followed by halogenation with a suitable halogenated succinimide followed by nucleophilic substitution with an amine of formula (V). The substitution reaction may be carried out in a suitable solvent, such as acetonitrile, dichloromethane, tetrahydrofuran, 1,4-dioxane or dimethylformamide; in the presence of an inorganic base such as K₂CO₃, Cs₂CO₃ or NaH, or an organic base such as triethylamine or diisopropylethylamine, at a suitable temperature comprised between room temperature and the reflux temperature (Step 2b). A compound of formula (Ia) may be then prepared by reacting a compound of formula (VIa) with a boronic acid of formula (VII) in the conditions described above for Step 3 in Method A (Step 3b).

A further alternative process for the preparation of a compound of formula (I) involves the reaction of a pyrazole of formula (X) with a compound of formula (III) followed by a reaction with an amine of formula (V) following the conditions described above for Steps 1 and 2 in Method A, representing a change of order of the steps described in Scheme 1.

A further alternative process for the preparation of a compound of formula (I) involves the reaction of a pyrazole of formula (II) with a compound of formula (XI) with the conditions described above for Step 1 in Method A; followed by reaction with a compound of formula (VII) and by a reaction with a compound of formula (XIII) following the conditions described above.

### Method C

Method C represents a process for synthetizing compounds according to general formula (I′a).

Thus, a process is described for the preparation of a compound of general formula (I′a):

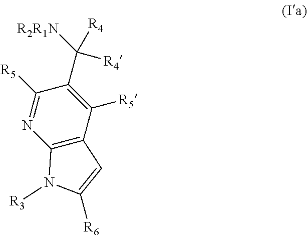

comprising the reaction of a compound of formula (XV):

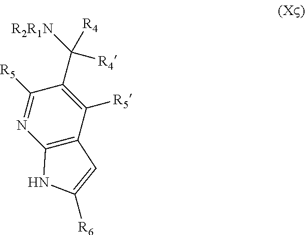

with a compound of formula (XVI):

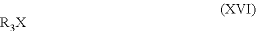

wherein R₁, R₂, R₃, R₄, R₄′, R₅, R₅′, R₆ are as defined along the detailed description and X is a halogen atom.

The compounds of formula (I′a) may be prepared by the process described in Scheme 3.

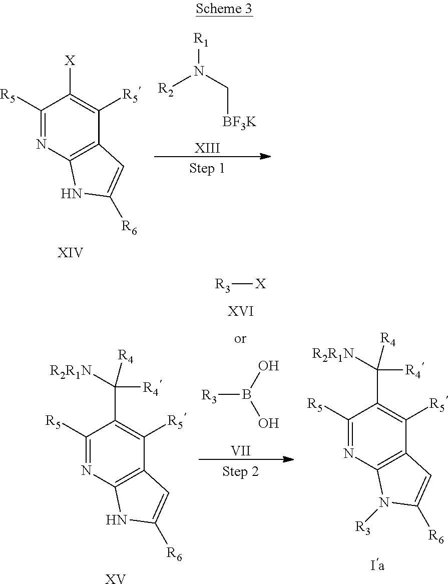

The process can be carried out as described below:

Step 1: A compound of formula (XV) can be prepared by treating a compound of formula (XIV) with a compound of formula (XIII) using the conditions described above for Step 3a in Method B.

Step 2: A compound of formula (I′a) can be prepared by treating a compound of formula (XV) with an aryl halide of formula (XVI) in the presence of a Cu or a Pd catalyst, such as copper iodide or palladium dibenzylideneacetone and a suitable base, such as cesium carbonate, optionally in the presence of a phosphine, such as XantPhos in a suitable solvent, such as dimethylformamide or dioxane, at a suitable temperature, preferably 110° C. Alternatively, it can also be prepared by treating a compound of formula (XV) with an aryl boronic acid of formula (VII) in the presence of a Cu catalyst, such as copper acetate and a suitable organic base, such as triethylamine, in a suitable solvent, such as dichloroethane, at a suitable temperature, preferably room temperature.

### Method D

Method D represents a process for synthetizing compounds according to general formula (I′b).

Thus, a process is described for the preparation of a compound of general formula (I′b):

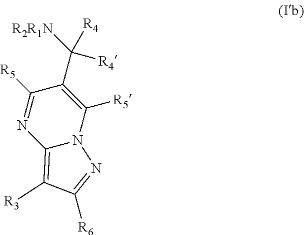

comprising the reaction of a compound of formula (XIX):

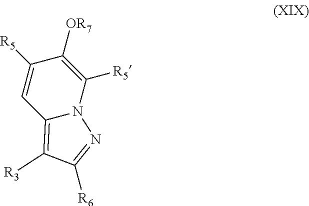

with a compound of formula (XIII):

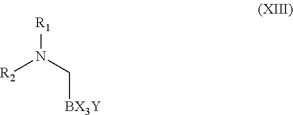

wherein R₁, R₂, R₃, R₄, R_{4′}, R₅, R_{5′} and R₆ are as defined along the detailed description, X represents a halogen atom, Y is an alkaline atom and R₇ represents a H atom, an alkyl radical or an alkylsulfonate radical.

The compounds of formula (I′b) may be prepared by the process described in Scheme 4:

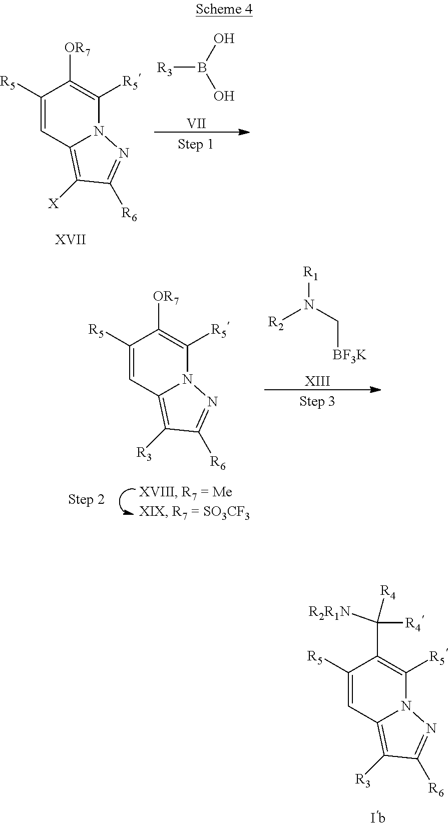

The process can be carried out as described below:

Step 1: A compound of formula (XVIII) is obtained by the reaction of a compound of formula (XVII) with a compound of formula (VIII) with the conditions described above for Step 3 in Method A.

Step 2: A compound of formula (XIX) can be prepared by treating a compound of formula (XVII) with boron tribromide in a suitable solvent, such as DCM, at a suitable temperature, preferably 40° C.; followed by treatment with a suitable activating agent such as trifluoromethanesulfonic anhydride, with a suitable base, such as triethylamine, in a suitable solvent, such as DCM, at a suitable temperature, preferably at 0° C.

Step 3: A compound of formula (I′b) can be prepared by treating a compound of formula (XIX) with a compound of formula (XIII) with the conditions described above for Step 3a in Method B.

### Method E

Method E represents a process for synthetizing compounds according to general formula (I′c).

Thus, a process is described for the preparation of a compound of general formula (I′c):

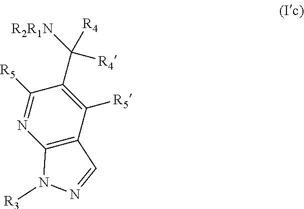

comprising the reaction of a compound of formula (XXI):

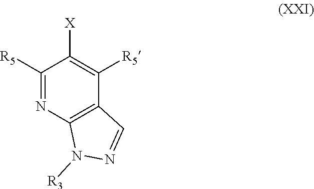

with a compound of formula (XIII):

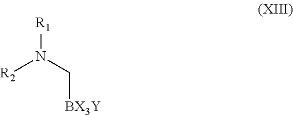

wherein R₁, R₂, R₃, R₄, R_{4′}, R₅, R_{5′} and R₆ are as defined along the detailed description, X represents a halogen atom and Y is an alkaline atom

The compounds of formula (I′c) may be prepared by the process described in Scheme 5:

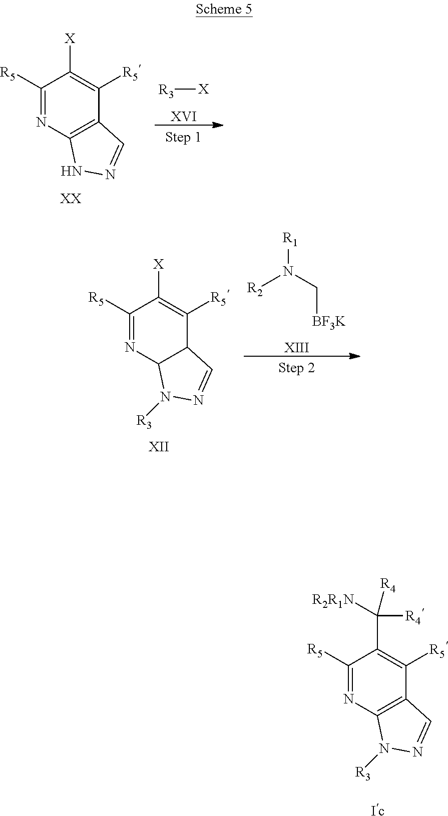

The process can be carried out as described below:

Step 1: A compound of formula (XXI) is obtained by reacting a compound of formula (XX) with a compound of formula (XVI) with a suitable base, such as NaH, in a suitable solvent, such as DMF, at a suitable temperature, preferably at 100° C.

Step 2: A compound of formula (I′c) can be prepared by treating a compound of formula (XXI) with a compound of formula (XIII) with the conditions described above for Step 3a in Method B.

The compounds of formula (II), (III), (V), (VII), (VIII), (XI), (XIII), XIV, XVI, XVII and XX used in the methods disclosed above are commercially available or can be synthesized following common procedures described in the literature and exemplified in the synthesis of some intermediates.

Turning to another aspect, the invention also relates to the therapeutic use of the compounds of general formula (I′). As mentioned above, compounds of general formula (I′) show a strong affinity to sigma receptors, especially to sigma-1 receptors and can behave as agonists, antagonists, inverse agonists, partial antagonists or partial agonists thereof. Therefore, compounds of general formula (I′) are useful as medicaments.

They are suitable for the treatment and/or prophylaxis of diseases and/or disorders mediated by sigma receptors and preferably by sigma-1 receptors. In this sense, compounds of formula (I′) are suitable for the treatment and/or prophylaxis of pain, especially neuropathic pain, inflammatory pain, and chronic pain or other pain conditions involving allodynia and/or hyperalgesia, or CNS disorder or diseases, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

The compounds of general formula (I′) are especially suited for the treatment of pain, especially neuropathic pain, inflammatory pain or other pain conditions involving allodynia and/or hyperalgesia or CNS disorder or diseases, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

Pain is defined by the International Association for the Study of Pain (IASP) as “an unpleasant sensory and emotional experience associated with actual or potential tissue damage or described in terms of such damage” (IASP, Classification of chronic pain, 2nd Edition, IASP Press (2002), 210). Even though pain is always subjective its causes or syndromes can be classified.

In a preferred embodiment compounds of the invention are used for the treatment and/or prophylaxis of allodynia and more specifically mechanical or thermal allodynia.

In another preferred embodiment compounds of the invention are used for the treatment and/or prophylaxis of hyperalgesia.

In yet another preferred embodiment the compounds of the invention are used for the treatment and/or prophylaxis of neuropathic pain and more specifically for the treatment and/or prophylaxis of hyperpathia.

A related aspect of the invention refers to the use of compounds of general formula (I′) for the manufacture of a medicament for the treatment and/or prophylaxis of disorders and diseases mediated by sigma receceptors and more preferably by sigma-1 receptors, as explained before.

Another related aspect of the invention refers to a method for the treatment and/or prophylaxis of disorders and diseases mediated by sigma receceptors and more preferably by sigma-1 receptors, as explained before comprising the administration of a therapeutically effective amount of a compound of general formula (I′) to a subject in need thereof.

Another aspect of the invention is a pharmaceutical composition, which comprises at least a compound of general formula (I′) or a pharmaceutically acceptable salt, isomer, co-crystal, prodrug or solvate thereof, and at least a pharmaceutically acceptable carrier, additive, adjuvant or vehicle.

The pharmaceutical composition of the invention can be formulated as a medicament in different pharmaceutical forms comprising at least a compound binding to the sigma receptor and optionally at least one further active substance and/or optionally at least one auxiliary substance.

The auxiliary substances or additives can be selected among carriers, excipients, support materials, lubricants, fillers, solvents, diluents, colorants, flavour conditioners such as sugars, antioxidants and/or agglutinants. In the case of suppositories, this may imply waxes or fatty acid esters or preservatives, emulsifiers and/or carriers for parenteral application. The selection of these auxiliary materials and/or additives and the amounts to be used will depend on the form of application of the pharmaceutical composition.

The pharmaceutical composition in accordance with the invention can be adapted to any form of administration, be it orally or parenterally, for example pulmonarily, nasally, rectally and/or intravenously.

Preferably, the composition is suitable for oral or parenteral administration, more preferably for oral, intravenous, intraperitoneal, intramuscular, subcutaneous, intrathekal, rectal, transdermal, transmucosal or nasal administration.

The composition of the invention can be formulated for oral administration in any form preferably selected from the group consisting of tablets, dragees, capsules, pills, chewing gums, powders, drops, gels, juices, syrups, solutions and suspensions. The composition of the present invention for oral administration may also be in the form of multiparticulates, preferably microparticles, microtablets, pellets or granules, optionally compressed into a tablet, filled into a capsule or suspended in a suitable liquid. Suitable liquids are known to those skilled in the art.

Suitable preparations for parenteral applications are solutions, suspensions, reconstitutable dry preparations or sprays.

The compounds of the invention can be formulated as deposits in dissolved form or in patches, for percutaneous application.

Skin applications include ointments, gels, creams, lotions, suspensions or emulsions.

The preferred form of rectal application is by means of suppositories.

In a preferred embodiment, the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the apropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

The following examples are merely illustrative of certain embodiments of the invention and cannot be considered as restricting it in any way.

### EXAMPLES

### Examples

The following abbreviations are used in the examples:
ACN: acetonitrile
Anh: anhydrous
Aq: aqueous
Dba: dibenzylideneacetone
DCM: dichloromethane
DMF: dimethylformamide
Dppf: 1,1′-Bis(diphenylphosphino)ferrocene
DME: dimethoxyethane
EtOAc: ethyl acetate
EtOH: ethanol
EX: example
h: hours
HPLC: high performance liquid chromatography
MeOH: methanol
MS: mass spectrometry
Min: minutes
RuPhos: 2-dicyclohexylphosphino-2′,6′-diisopropoxybiphenyl
Rt.: retention time
r.t.: room temperature
Sat: saturated
Sol: solution
TEA: triethylamine
Tf₂O: Trifluoromethanesulfonic anhydride
TFA: trifluoroacetic acid
THF: tetrahydrofuran
^{t}BuOK: potassium tert-butoxide
XantPhos: 4,5-bis(diphenylphosphino)-9,9-dimethylxanthene
XPhos: 2-dicyclohexylphosphino-2′,4′,6′-triisopropylbiphenyl

The following methods were used to determine the HPLC-MS spectra:

Method A: Column ZORBAX Extend-C18 RRHD 2.1×50 mm, 1.8 μm, flow rate 0.61 mL/min; A: NH₄HCO₃ 10 mM, B: ACN; gradient 0.3 min 98% A, 98% A to 100% B in 2.65 min; isocratic 2.05 min 100% B.

Method B: Column ZORBAX Extend-C18 RRHD 2.1×50 mm, 1.8 μm, flow rate 0.61 mL/min; A: NH₄HCO₃ 10 mM, B: ACN, C: MeOH+0.1% formic acid; gradient 0.3 min 98% A, 98% A to 0:95:5 A:B:C in 2.7 min; 0:95:5 A:B:C to 100% B in 0.1 min; isocratic 2 min 100% B.

### Synthesis of Examples

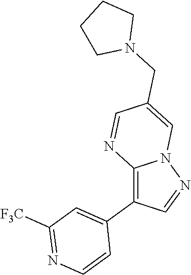 [SMILES: FC(F)(F)c1cc(-c2cnn3cc(CN4CCCC4)cnc23)ccn1]

### Example 1

6-(Pyrrolidin-1-ylmethyl)-3-(2-(trifluoromethyl)pyridin-4-yl) pyrazolo[1,5-a]pyrimidine
Step a. 3-Bromopyrazolo[1,5-a]pyrimidine-6-carbaldehyde

A solution of 4-bromo-1H-pyrazol-5-amine (6.0 g, 37 mmol) and methanetricarbaldehyde (3.7 g, 37 mmol) in EtOH was heated at 80° C. for 16 h. After this time, solvent was removed under reduced pressure and the residue was diluted with EtOAc and washed with aq sat NaHCO₃. The organic layer was dried over anh Na₂SO₄, filtered and concentrated to dryness. The residue was diluted with DCM (100 mL) and TFA (20 mL) and the mixture was stirred at r.t. for 16 h more. The solvent was removed under reduced pressure and the residue was diluted with DCM and washed with aq sat NaHCO₃ and brine. The organic layer was dried over anh Na₂SO₄, filtered and concentrated to dryness to give the title compound (5.9 g, Yield: 96%).

HPLC-MS (Method A): Rt. 1.29 min; ESI⁺-MS m/z: 226.0 (M+1).

Step b. 3-Bromo-6-(pyrrolidin-1-ylmethyl)pyrazolo[1,5-a]pyrimidine

Pyrrolidine (0.2 mL, 2.45 mmol) was added to a solution of the compound obtained in step a (370 mg, 1.64 mmol) in DCM (22 mL) and the reaction was stirred at r.t. for 15 min. Then, NaBH(OAc)₃ (520 mg, 2.45 mmol) was added and the reaction was stirred for 16 h more. After this time, the mixture was washed with water and brine. The organic layer was dried over anh Na₂SO₄, filtered and concentrated to dryness. The crude product was purified by flash chromatography, silica gel, Chx:EtOAc to give the title compound (317 mg, Yield: 69%).

HPLC-MS (Method A): Rt. 1.55 min; ESI⁺-MS m/z: 281.0 (M+1).

Step c. Title Compound

A solution of the compound obtained in step b (54 mg, 0.19 mmol), (2-(trifluoromethyl) pyridin-4-yl)boronic acid (55 mg, 0.29 mmol), Pd(PPh₃)₄ (12 mg, 0.01 mmol) and K₂CO₃ (53 mg, 0.38 mmol) in DME:H₂O was heated under MW irradiation at 130° C. for 15 min. After this time, solvent was removed under reduced pressure and the residue was diluted with EtOAc and washed with aq sat NaHCO₃. The organic layer was dried over anh Na₂SO₄, filtered and concentrated to dryness. The crude product was purified by flash chromatography, silica gel, DCM:MeOH to give the title compound (44 mg, Yield: 66%).

HPLC-MS (Method A): Rt. 2.03 min; ESI⁺-MS m/z: 348.0 (M+1).

This method was used for the preparation of examples 2-12 using suitable starting materials:

| | | | | MS | |
|---|---|---|---|---|---|
| | | CHEMICAL | Rt | (M + | HPLC |
| STRUCTURE | EX | NAME | (min) | H) | Method |
| 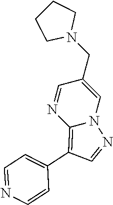 [SMILES: c1cc(-c2cnn3cc(CN4CCCC4)cnc23)ccn1] | 2 | 3-(Pyridin- 4-yl)-6- (pyrrolidin-1- ylmethyl) pyrazolo[1,5- a]pyrimidine | 1.49 | 280.2 | A |
| 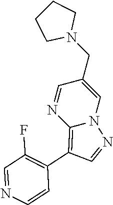 [SMILES: Fc1cnccc1-c1cnn2cc(CN3CCCC3)cnc12] | 3 | 3-(3- Fluoropyridin- 4-yl)- 6-(pyrrolidin-1- ylmethyl) pyrazolo[1,5- a]pyrimidine | 1.67 | 298.0 | A |
| 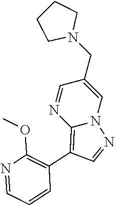 [SMILES: COc1ncccc1-c1cnn2cc(CN3CCCC3)cnc12] | 4 | 3-(2- Methoxypyridin- 3-yl)-6- (pyrrolidin-1- ylmethyl) pyrazolo[1,5- a]pyrimidine | 1.83 | 310.0 | B |
| 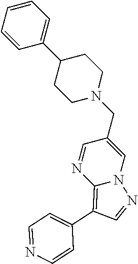 [SMILES: c1ccc(C2CCN(Cc3cnc4c(-c5ccncc5)cnn4c3)CC2)cc1] | 5 | 6-((4- Phenylpiperidin- 1-yl)methyl)-3- (pyridin-4- yl)pyrazolo[1,5- a]pyrimidine | 2.20 | 370.2 | A |
| 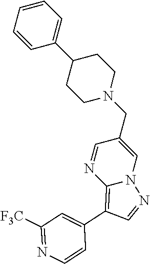 [SMILES: FC(F)(F)c1cc(-c2cnn3cc(CN4CCC(c5ccccc5)CC4)cnc23)ccn1] | 6 | 6-((4- Phenylpiperidin- 1- yl)methyl)-3-(2- (trifluoromethyl) pyridin- 4-yl)pyrazolo [1,5- a]pyrimidine | 2.61 | 438.2 | A |
| 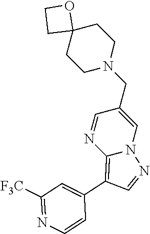 [SMILES: FC(F)(F)c1cc(-c2cnn3cc(CN4CCC5(CCO5)CC4)cnc23)ccn1] | 7 | 7-((3-(2- (Trifluoromethyl) pyridin- 4-yl)pyrazolo [1,5-a] pyrimidin-6-yl) methyl)- 1-oxa-7- azaspiro[3.5] nonane | 1.84 | 404.0 | B |
| 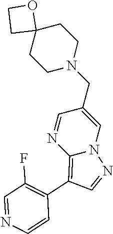 [SMILES: Fc1cnccc1-c1cnn2cc(CN3CCC4(CCO4)CC3)cnc12] | 8 | 7-((3-(3- Fluoropyridin- 4- yl)pyrazolo[1,5- a]pyrimidin-6- yl)methyl)- 1-oxa-7- azaspiro[3.5] nonane | 1.52 | 354.2 | A |
| 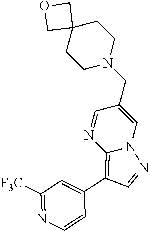 [SMILES: FC(F)(F)c1cc(-c2cnn3cc(CN4CCC5(CC4)COC5)cnc23)ccn1] | 9 | 7-((3-(2- (Trifluoromethyl) pyridin- 4-yl)pyrazolo [1,5-a] pyrimidin-6- yl)methyl)- 2-oxa-7- azaspiro[3.5] nonane | 1.85 | 404.2 | A |
| 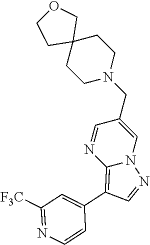 [SMILES: FC(F)(F)c1cc(-c2cnn3cc(CN4CCC5(CCOC5)CC4)cnc23)ccn1] | 10 | 8-((3-(2- (Trifluoromethyl) pyridin- 4-yl)pyrazolo [1,5- a]pyrimidin-6- yl)methyl)- 2-oxa-8- azaspiro[4.5] decane | 1.83 | 404.0 | A |
| 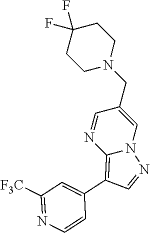 [SMILES: FC1(F)CCN(Cc2cnc3c(-c4ccnc(C(F)(F)F)c4)cnn3c2)CC1] | 11 | 6-((4,4- Difluoropiperidin- 1-yl)methyl)- 3-(2- (trifluoromethyl) pyridin- 4-yl)pyrazolo [1,5- a]pyrimidine | 2.20 | 398.2 | A |
| 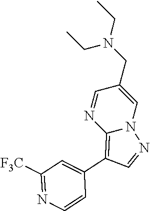 [SMILES: CCN(CC)Cc1cnc2c(-c3ccnc(C(F)(F)F)c3)cnn2c1] | 12 | N-Ethyl-N- ((3-(2- (trifluoromethyl) pyridin- 4-yl)pyrazolo [1,5- a]pyrimidin- 6- yl)methyl) ethanamine | 2.28 | 350.2 | A |

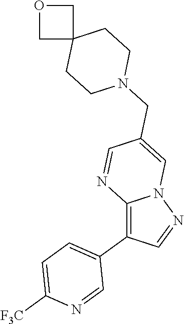 [SMILES: FC(F)(F)c1ccc(-c2cnn3cc(CN4CCC5(CC4)COC5)cnc23)cn1]

### Example 13

7-((3-(6-(Trifluoromethyl)pyridin-3-yl)pyrazolo[1,5-a]pyrimidin-6-yl) methyl)-2-oxa-7-azaspiro[3.5]nonane
Step a. 7-((3-Bromopyrazolo[1,5-a]pyrimidin-6-yl)methyl)-2-oxa-7-azaspiro[3.5]nonane

Starting from the product obtained in step a of Example 1 (4.97 g, 22 mmol) and 2-oxa-7-azaspiro[3.5]nonane (3.63 g, 29 mmol) and following the experimental procedure described in step b of Example 1, the title compound was obtained (6.57 g, Yield: 89%).

HPLC-MS (Method A): Rt. 1.44 min; ESI+-MS m/z: 337.0 (M+H).

Step b. Title Compound

A solution of the compound obtained in step a (80 mg, 0.24 mmol), 5-(4,4,5,5-tetramethyl-1, 3,2-dioxaborolan-2-yl)-2-(trifluoromethyl)pyridine (50 mg, 0.26 mmol), Pd(dppf)₂Cl₂ (17 mg, 0.024 mmol) and Cs₂CO₃ (232 mg, 0.7 mmol) in dioxane under Ar atmosphere was heated at 110° C. for 16 h. After this time, the solvent was removed under reduced pressure and the residue was diluted with EtOAc and washed with aq sat NaHCO₃. The organic layer was dried over anh Na₂SO₄, filtered and concentrated to dryness. The crude product was purified by flash chromatography, silica gel, DCM:MeOH to give the title compound (57 mg, Yield: 59%).

HPLC-MS (Method B): Rt. 1.85 min; ESI⁺-MS m/z: 404.0 (M+1).

This method was used for the preparation of examples 14-20 using suitable starting materials:

| | | CHEMICAL | Rt | MS | HPLC |
|---|---|---|---|---|---|
| STRUCTURE | EX | NAME | (min) | (M + H) | Method |
| 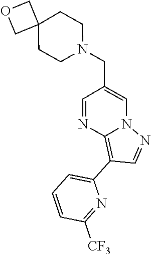 [SMILES: FC(F)(F)c1cccc(-c2cnn3cc(CN4CCC5(CC4)COC5)cnc23)n1] | 14 | 7-((3-(6- (Trifluoromethyl) pyridin- 2-yl)pyrazolo[1,5- a]pyrimidin-6- yl)methyl)- 2-oxa-7- azaspiro[3.5] nonane | 2.05 | 404.2 | B |
| 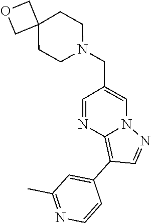 [SMILES: Cc1cc(-c2cnn3cc(CN4CCC5(CC4)COC5)cnc23)ccn1] | 15 | 7-((3-(2- Methylpyridin- 4-yl)pyrazolo [1,5-a] pyrimidin-6- yl)methyl)- 2-oxa-7- azaspiro[3.5] nonane | 1.40 | 350.2 | B |
| 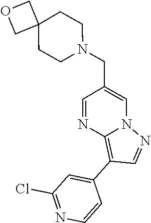 [SMILES: Clc1cc(-c2cnn3cc(CN4CCC5(CC4)COC5)cnc23)ccn1] | 16 | 7-((3-(2- Chloropyridin- 4-yl)pyrazolo [1,5- a]pyrimidin- 6-yl)methyl)- 2-oxa-7- azaspiro[3.5] nonane | 1.57 | 370.2 | A |
| 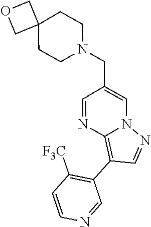 [SMILES: FC(F)(F)c1ccncc1-c1cnn2cc(CN3CCC4(CC3)COC4)cnc12] | 17 | 7-((3-(4- (Trifluoromethyl) pyridin- 3-yl)pyrazolo [1,5- a]pyrimidin- 6-yl)methyl)- 2-oxa-7- azaspiro[3.5] nonane | 1.57 | 404.2 | B |
| 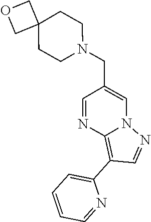 [SMILES: c1ccc(-c2cnn3cc(CN4CCC5(CC4)COC5)cnc23)nc1] | 18 | 7-((3-(Pyridin-2- yl)pyrazolo[1,5- a]pyrimidin-6- yl)methyl)- 2-oxa-7- azaspiro[3.5] nonane | 1.47 | 336.2 | A |
| 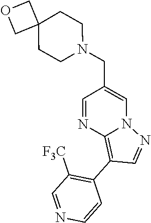 [SMILES: FC(F)(F)c1cnccc1-c1cnn2cc(CN3CCC4(CC3)COC4)cnc12] | 19 | 7-((3-(3- (Trifluoromethyl) pyridin- 4-yl)pyrazolo [1,5- a]pyrimidin-6- yl)methyl)- 2-oxa-7- azaspiro[3.5] nonane | 1.59 | 404.2 | B |
| 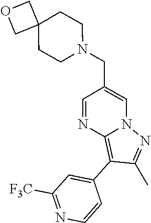 [SMILES: Cc1nn2cc(CN3CCC4(CC3)COC4)cnc2c1-c1ccnc(C(F)(F)F)c1] | 20 | 7-((2-Methyl- 3-(2- (trifluoromethyl) pyridin- 4-yl)pyrazolo [1,5- a]pyrimidin-6- yl)methyl)- 2-oxa-7- azaspiro[3.5] nonane | 1.85 | 418.2 | B |
| 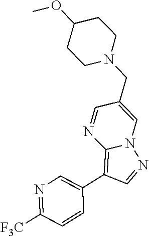 [SMILES: COC1CCN(Cc2cnc3c(-c4ccc(C(F)(F)F)nc4)cnn3c2)CC1] | 21 | 6-((4- Methoxypiperidin- 1-yl)methyl)-3-(6- (trifluoromethyl) pyridin- 3-yl)pyrazolo[1,5- a]pyrimidine | 2.01 | 391.9 | B |
| 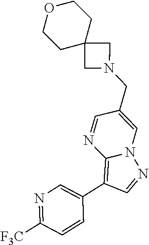 [SMILES: FC(F)(F)c1ccc(-c2cnn3cc(CN4CC5(CCOCC5)C4)cnc23)cn1] | 22 | 2-((3-(6- (Trifluoromethyl) pyridin- 3-yl)pyrazolo[1,5- a]pyrimidin- 6-yl)methyl)- 7-oxa-2- azaspiro[3.5] nonane | 1.90 | 404.2 | A |
| 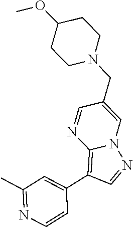 [SMILES: COC1CCN(Cc2cnc3c(-c4ccnc(C)c4)cnn3c2)CC1] | 23 | 6-((4- Methoxypiperidin- 1-yl)methyl)-3-(2- methylpyridin-4- yl)pyrazolo[1,5- a]pyrimidine | 1.51 | 337.9 | B |
| 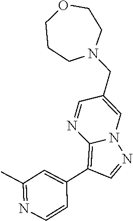 [SMILES: Cc1cc(-c2cnn3cc(CN4CCCOCC4)cnc23)ccn1] | 24 | 4-((3-(2- Methylpyridin-4- yl)pyrazolo[1,5- a]pyrimidin-6- yl)methyl)- 1,4-oxazepane | 1.49 | 324.2 | A |
| 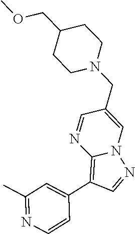 [SMILES: COCC1CCN(Cc2cnc3c(-c4ccnc(C)c4)cnn3c2)CC1] | 25 | 6-((4- (Methoxymethyl) piperidin- 1-yl)methyl)-3-(2- methylpyridin-4- yl)pyrazolo[1,5- a]pyrimidine | 1.71 | 352.2 | A |
| 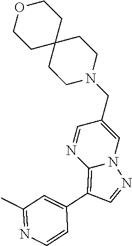 [SMILES: Cc1cc(-c2cnn3cc(CN4CCC5(CCOCC5)CC4)cnc23)ccn1] | 26 | 9-((3-(2- Methylpyridin- 4- yl)pyrazolo [1,5- a]pyrimidin- 6-yl)methyl)- 3-oxa-9- azaspiro[5.5] undecane | 1.66 | 378.2 | A |
| 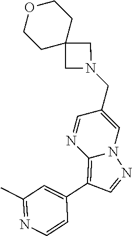 [SMILES: Cc1cc(-c2cnn3cc(CN4CC5(CCOCC5)C4)cnc23)ccn1] | 27 | 2-((3-(2- Methylpyridin- 4-yl)pyrazolo [1,5-a] pyrimidin-6- yl)methyl)- 7-oxa-2- azaspiro[3.5] nonane | 1.42 | 350.1 | B |

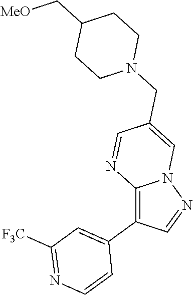 [SMILES: COCC1CCN(Cc2cnc3c(-c4ccnc(C(F)(F)F)c4)cnn3c2)CC1]

### Example 28

6-((4-(Methoxymethyl)piperidin-1-yl)methyl)-3-(2-(trifluoromethyl) pyridin-4-yl)pyrazolo[1,5-a]pyrimidine

### Step a. 2-(2-(Trifluoromethyl)pyridin-4-yl)acetonitrile

A 2.5 M solution of n-BuLi in hexane (79.6 mL, 199 mmol) was slowly added to anh THF at −78° C. Anh ACN (10.5 mL, 199 mmol) was added dropwise and the mixture stirred for 30 min. A solution of 4-bromo-2-(trifluoromethyl)pyridine (15 g, 66.4 mmol) in anh THF was added dropwise at −78° C. and the mixture was stirred at −30° C. during 40 min more. After this time, the reaction was quenched with aq sat NaHCO₃, solvent was removed under reduced pressure and the residue was diluted with DCM and washed with aq sat NaHCO₃ and brine. The organic layer was dried over anh Na₂SO₄, filtered and concentrated to dryness to give the title compound (21 g, Yield: Quant.).

HPLC (Method A): Rt. 1.46 min; MS: 185.0 (M-H).

### Step b. (E)-3-(Dimethylamino)-2-(2-(trifluoromethyl)pyridin-4-yl)acrylonitrile

To a solution of the compound obtained in step a (2.48 g, 13.3 mmol) in toluene (60 mL), 1,1-dimethoxy-N,N-dimethylmethanamine (2.65 mL, 20 mmol) was added. The reaction mixture was heated at 90° C. for 90 min. All volatiles were removed under reduced pressure to give the title compound (2.82 g, Yield: 88%).

HPLC (Method A): Rt. 1.73 min; MS: 242.0 (M+H).

Step c. 4-(2-(Trifluoromethyl)pyridin-4-yl)-1H-pyrazol-5-amine

A solution of the compound obtained in step b (2.82 g, 11.7 mmol) and hydrazine hydrate (2.26 mL, 46.7 mmol) in EtOH:H₂O (8:1) (67.5 mL) was heated at 80° C. for 16 h. After this time, the solvent was removed under reduced pressure and the residue was suspended in cold water and filtered. The solid was dried under vacuum at 45° C. during 16 h to give the title compound (1.04 g, Yield: 40%).

HPLC (Method A): Rt.1.23 min; MS: 229.0 (M+H).

### Step d. 6-Bromo-3-(2-(trifluoromethyl)pyridin-4-yl)pyrazolo[1,5-a]pyrimidine

A solution of the compound obtained in step c (4.0 g, 17.5 mmol) and 2-bromomalonaldehyde (2.65 g, 17.5 mmol) in EtOH (67.5 mL) was heated at 80° C.for 3 h. The solution was cooled to r.t. and the suspension was filtered washing with cold EtOH. The solid was dried under vacuum at 45° C. during 16 h to give the title compound (4.09 g, Yield: 68%).

HPLC-MS (Method A): Rt. 2.11 min; ESI+-MS m/z: 342.8 (M+1).

Step e. Potassium trifluoro((4-(methoxymethyl)piperidin-1-yl)methyl)borate

A schlenk flask charged with 4-(methoxymethyl)piperidine (359 mg, 2.78 mmol) and potassium (bromomethyl)trifluoroborate (558 mg, 2.78 mmol), was evacuated and backfilled with argon. THF:^{t}BuOH (2:1, 8 mL), degassed by means of bubbling argon to the solution for 5 min, was added and the reaction mixture was heated at 80° C.overnight. The mixture was dried under vacuum and the crude product was suspended in Et₂O and filtered to give the title compound (687 mg, Yield: 99%).

HPLC-MS (Method A): Rt. 0.87 min; ESI⁺-MS m/z: 210.1 (M-K).

Step f. Title Compound

A sealed tube charged with the compound obtained in the step d (80 mg, 0.23 mmol), the compound obtained in step e (84 mg, 0.34 mmol), Pd(OAc)₂ (9 mg, 0.04 mmol), XPhos (39 mg, 0.8 mmol) and Cs₂CO₃ (221 mg, 0.68 mmol), was evacuated and backfilled with argon. Dioxane:H₂O (9:1, 4.5 mL), degassed by means of bubbling argon to the solution for 5 min, was added and the reaction mixture was stirred at 110° C. overnight. The solvent was removed under vacuum, the residue was dissolved in EtOAc and washed with aq NaHCO₃ sat sol. The combined organic layers were dried over Na₂SO₄, filtered, and concentrated under vacuum. The crude product was purified by flash chromatography, silica gel, DCM:MeOH to give the title compound (31 mg, Yield: 33%).

HPLC-MS (Method A): Rt. 2.14 min; ESI⁺-MS m/z: 406.2 (M+1).

This method was used for the preparation of examples 29-35 using suitable starting materials:

| | | | | MS | |
|---|---|---|---|---|---|
| | | CHEMICAL | Rt | (M + | HPLC |
| STRUCTURE | EX | NAME | (min) | H) | Method |
| 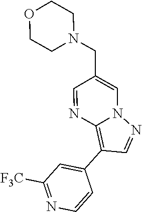 [SMILES: FC(F)(F)c1cc(-c2cnn3cc(CN4CCOCC4)cnc23)ccn1] | 29 | 4-((3-(2- (Trifluoromethyl) pyridin- 4-yl)pyrazolo[1,5- a]pyrimidin-6- yl)methyl) morpholine | 1.79 | 364.0 | A |
| 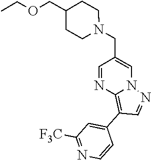 [SMILES: CCOCC1CCN(Cc2cnc3c(-c4ccnc(C(F)(F)F)c4)cnn3c2)CC1] | 30 | 6-((4- (Ethoxymethyl) piperidin- 1-yl)methyl)-3-(2- (trifluoromethyl) pyridin- 4-yl)pyrazolo[1,5- a]pyrimidine | 2.32 | 420.2 | A |
| 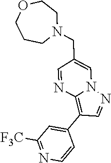 [SMILES: FC(F)(F)c1cc(-c2cnn3cc(CN4CCCOCC4)cnc23)ccn1] | 31 | 4-((3-(2- (Trifluoromethyl) pyridin- 4-yl)pyrazolo[1,5- a]pyrimidin-6- yl)methyl)- 1,4-oxazepane | 1.87 | 378.1 | B |
| 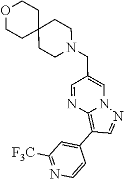 [SMILES: FC(F)(F)c1cc(-c2cnn3cc(CN4CCC5(CCOCC5)CC4)cnc23)ccn1] | 32 | 9-((3-(2- (Trifluoromethyl) pyridin- 4-yl)pyrazolo[1,5- a]pyrimidin-6- yl)methyl)- 3-oxa-9- azaspiro[5.5] undecane | 2.07 | 432.2 | B |
| 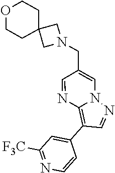 [SMILES: FC(F)(F)c1cc(-c2cnn3cc(CN4CC5(CCOCC5)C4)cnc23)ccn1] | 33 | 2-((3-(2- (Trifluoromethyl) pyridin- 4-yl)pyrazolo [1,5- a]pyrimidin-6- yl)methyl)- 7-oxa-2- azaspiro[3.5] nonane | 1.84 | 404.2 | B |
| 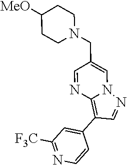 [SMILES: COC1CCN(Cc2cnc3c(-c4ccnc(C(F)(F)F)c4)cnn3c2)CC1] | 34 | 6-((4- Methoxypiperidin- 1-yl)methyl)-3-(2- (trifluoromethyl) pyridin- 4-yl)pyrazolo[1,5- a]pyrimidine | 1.99 | 392.0 | A |
| 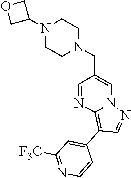 [SMILES: FC(F)(F)c1cc(-c2cnn3cc(CN4CCN(C5COC5)CC4)cnc23)ccn1] | 35 | 6-((4-(Oxetan-3- yl)piperazin-1- yl)methyl)- 3-(2- (trifluoromethyl) pyridin- 4-yl)pyrazolo[1,5- a]pyrimidine | 1.65 | 419.0 | A |

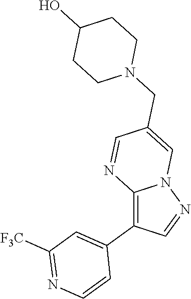 [SMILES: OC1CCN(Cc2cnc3c(-c4ccnc(C(F)(F)F)c4)cnn3c2)CC1]

### Example 36

1-((3-(2-(Trifluoromethyl)pyridin-4-yl)pyrazolo[1,5-a]pyrimidin-6-yl) methyl)piperidin-4-ol

### Step a. (3-(2-(Trifluoromethyl)pyridin-4-yl)pyrazolo[1,5-a]pyrimidin-6-yl)methanol

A sealed tube charged with the compound obtained in the step d of Example 28 (200 mg, 0.58 mmol), potassium (acetoxymethyl)trifluoroborate (107 mg, 0.76 mmol), Pd(dba)₂ (50 mg, 0.09 mmol), RuPhos (81 mg, 0.17 mmol) and Na₂CO₃ (92 mg, 0.9 mmol), was evacuated and backfilled with argon. Dioxane:H₂O (9:1, 11 mL), degassed by means of bubbling argon to the solution for 5 min, was added and the reaction mixture was stirred at 110° C. overnight. The solvent was removed under vacuum, the residue was dissolved in EtOAc and washed with aq NaHCO₃ sat sol. The combined organic layers were dried over Na₂SO₄, filtered, and concentrated under vacuum. The crude product was purified by flash chromatography, silica gel, DCM:MeOH to give the title compound (102 mg, Yield: 59%).

HPLC-MS (Method A): Rt. 1.60 min; ESI⁺-MS m/z: 295.0 (M+1).

Step b. Title Compound

Methanesulfonyl chloride (29 μL, 0.37 mmol) was added to a solution of the compound obtained in step a (100 mg, 0.34 mmol) and TEA (61 μL, 0.44 mmol) in ACN (10 mL) and the solution was stirred for 90 min. After this time, piperidin-4-ol (172 mg, 1.7 mmol) was added and the mixture was stirred during 2 h more. The reaction was quenched with aq sat NaHCO₃, solvent was removed under reduced pressure and the residue was diluted with DCM and washed with aq sat NaHCO₃ and brine. The organic layer was dried over anh Na₂SO₄, filtered and concentrated to dryness. The crude product was purified by flash chromatography, silica gel, DCM:MeOH to give the title compound (28 mg, Yield: 20%).

HPLC (Method A): Rt. 1.59 min; MS: 378.2 (M-H).

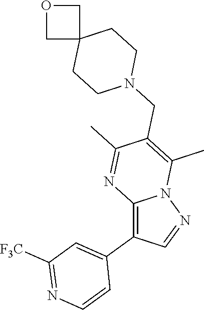 [SMILES: Cc1nc2c(-c3ccnc(C(F)(F)F)c3)cnn2c(C)c1CN1CCC2(CC1)COC2]

### Example 37

7-((5,7-Dimethyl-3-(2-(trifluoromethyl)pyridin-4-yl)pyrazolo[1,5-a]pyrimidin-6-yl)methyl)-2-oxa-7-azaspiro[3.5]nonane
Step a. 3-Bromo-6-chloro-5,7-dimethylpyrazolo[1,5-a]pyrimidine

A solution of 4-bromo-1H-pyrazol-5-amine (400 mg, 2.47 mmol) and 3-chloropentane-2,4-dione (0.28 mL, 2.47 mmol) in EtOH (5.4 mL) was heated at 80° C.for 16 h. The solution was cooled to r.t. and the solvent was removed under vacuum. The residue was dissolved in EtOAc and washed with aq NaHCO₃ sat sol. The organic layer was washed with brine, dried over Na₂SO₄, filtered, and concentrated under vacuum to give the title compound (564 mg, Yield: 88%).

HPLC-MS (Method A): Rt. 2.07 Min; ESI⁺-MS m/z: 261.8 (M+1).

Step b. 6-Chloro-5,7-dimethyl-3-(2-(trifluoromethyl)pyridine-4-yl)pyrazolo[1,5-a]pyrimidine

Starting from the product obtained in step a (50 mg, 0.19 mmol) and 4-(4,4,5,5-tetramethyl-1,3,2-dioxaborolan-2-yl)-2-(trifluoromethyl)pyridine (78 mg, 0.28 mmol) and following the experimental procedure described in step c of Example 1, the title compound was obtained (41 mg, Yield: 65%).

HPLC-MS (Method A): Rt. 2.45 Min; ESI⁺-MS m/z: 327.0 (M+1).

Step c. Potassium ((2-oxa-7-azaspiro[3.5]nonan-7-yl)methyl)trifluoroborate

Starting from 2-oxa-7-azaspiro[3.5]nonane (1.0 g, 7.86 mmol) and following the experimental procedure described in step e of Example 28, the title compound was obtained (2.26 g, Yield: Quant).

¹H NMR (400 MHZ, CD₃OD) δ 4.55 (s, 2H), 4.43 (s, 2H), 3.40-3.42 (m, 2H), 2.85 (t, J=13.0 Hz, 2H), 2.33-2.23 (s, 2H), 2.16-2.09 (m, 2H), 1.92 (td, J=14.3, 4.0 Hz, 2H).

Step d. 7-((5,7-Dimethyl-3-(2-(trifluoromethyl)pyridin-4-yl)pyrazolo[1,5-a]pyrimidin-6-yl) methyl)-2-oxa-7-azaspiro[3.5]nonane

Starting from the product obtained in step b (41 mg, 0.12 mmol) and the product obtained in step c (74 mg, 0.36 mmol) and following the experimental procedure described in step f of Example 28, the title compound was obtained (4 mg, Yield: 8%).

HPLC-MS (Method A): Rt. 2.38 Min; ESI⁺-MS m/z: 432.0 (M+1).

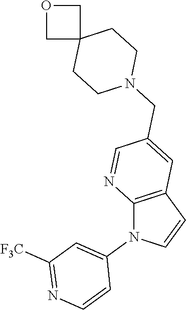 [SMILES: FC(F)(F)c1cc(-n2ccc3cc(CN4CCC5(CC4)COC5)cnc32)ccn1]

### Example 38

7-((1-(2-(Trifluoromethyl)pyridin-4-yl)-1H-pyrrolo[2,3-b]pyridin-5-yl) methyl)-2-oxa-7-azaspiro[3.5]nonane
Step a. 7-((1H-Pyrrolo[2,3-b]pyridin-5-yl)methyl)-2-oxa-7-azaspiro[3.5]nonane Starting from the product obtained in step c of Example 37 (158 mg, 0.76 mmol) and 5-bromo-1H-pyrrolo[2,3-b]pyridine (100 mg, 0.51 mmol) and following the experimental procedure described in step f of Example 28, the title compound was obtained (58 mg, Yield: 44%).

HPLC-MS (Method A): Rt. 1.25 min; ESI⁺-MS m/z: 258.2 (M+1).

Step b. Title Compound

A sealed tube charged with 4-bromo-2-(trifluoromethyl)pyridine (61 mg, 0.22 mmol), the compound obtained in step a (58 mg, 0.22 mmol), Cul (17 mg, 0.09 mmol) and Cs₂CO₃ (147 mg, 0.45 mmol), was evacuated and backfilled with argon. DMF (3 mL), degassed by means of bubbling argon to the solution for 5 min, was added and the reaction mixture was stirred at 110° C.overnight. The resulting mixture was filtered through Celite® and washed with EtOAc. The solvent was removed under vacuum and the residue was purified by flash chromatography, silica gel, DCM:MeOH to give the title compound (31 mg, Yield: 34%).

HPLC-MS (Method A): Rt. 2.06 min; ESI⁺-MS m/z: 403.0 (M+1).

This method was used for the preparation of examples 39-50 using suitable starting materials:

| | | | | MS | |
|---|---|---|---|---|---|
| | | CHEMICAL | Rt | (M + | HPLC |
| STRUCTURE | EX | NAME | (min) | H) | Method |
| 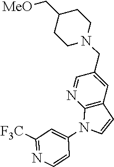 [SMILES: COCC1CCN(Cc2cnc3c(ccn3-c3ccnc(C(F)(F)F)c3)c2)CC1] | 39 | 5-((4- (Methoxymethyl) piperidin-1- yl)methyl)- 1-(2- (trifluoromethyl) pyridin- 4-yl)-1H- pyrrolo[2,3- b]pyridine | 2.33 | 405.2 | B |
| 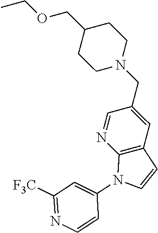 [SMILES: CCOCC1CCN(Cc2cnc3c(ccn3-c3ccnc(C(F)(F)F)c3)c2)CC1] | 40 | 5-((4- (Ethoxymethyl) piperidin- 1-yl)methyl)-1-(2- (trifluoromethyl) pyridin- 4-yl)-1H-pyrrolo [2,3- b]pyridine | 2.55 | 419.2 | A |
| 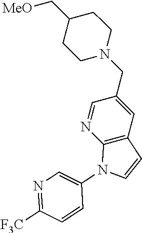 [SMILES: COCC1CCN(Cc2cnc3c(ccn3-c3ccc(C(F)(F)F)nc3)c2)CC1] | 41 | 5-((4- (Methoxymethyl) piperidin-1- yl)methyl)-1-(6- (trifluoromethyl) pyridin- 3-yl)-1H-pyrrolo [2,3- b]pyridine | 2.32 | 405.2 | B |
| 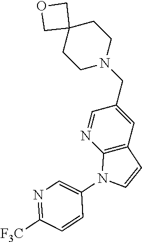 [SMILES: FC(F)(F)c1ccc(-n2ccc3cc(CN4CCC5(CC4)COC5)cnc32)cn1] | 42 | 7-((1-(6- (Trifluoromethyl) pyridin- 3-yl)-1H- pyrrolo[2,3- b]pyridin-5-yl) methyl)-2- oxa-7- azaspiro[3.5] nonane | 2.04 | 403.1 | B |
| 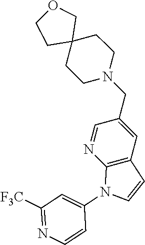 [SMILES: FC(F)(F)c1cc(-n2ccc3cc(CN4CCC5(CCOC5)CC4)cnc32)ccn1] | 43 | 8-((1-(2- (Trifluoromethyl) pyridin- 4-yl)-1H- pyrrolo[2,3- b]pyridin-5-yl) methyl)-2- oxa-8- azaspiro[4.5] decane | 2.21 | 417.2 | B |
| 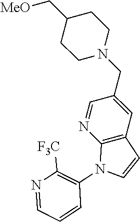 [SMILES: COCC1CCN(Cc2cnc3c(ccn3-c3cccnc3C(F)(F)F)c2)CC1] | 44 | 5-((4- (Methoxymethyl) piperidin-1- yl)methyl)-1-(2- (trifluoromethyl) pyridin- 3-yl)-1H- pyrrolo[2,3- b]pyridine | 2.00 | 405.0 | A |
| 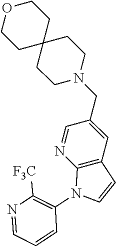 [SMILES: FC(F)(F)c1ncccc1-n1ccc2cc(CN3CCC4(CCOCC4)CC3)cnc21] | 45 | 9-((1-(2- (Trifluoromethyl) pyridin-3-yl)- 1H-pyrrolo[2,3- b]pyridin-5-yl) methyl)-3- oxa-9- azaspiro[5.5] undecane | 1.81 | 431.2 | B |
| 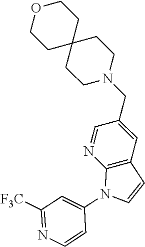 [SMILES: FC(F)(F)c1cc(-n2ccc3cc(CN4CCC5(CCOCC5)CC4)cnc32)ccn1] | 46 | 9-((1-(2- (Trifluoromethyl) pyridin- 4-yl)-1H- pyrrolo[2,3- b]pyridin-5-yl) methyl)-3- oxa-9- azaspiro[5.5] undecane | 2.27 | 431.0 | B |
| 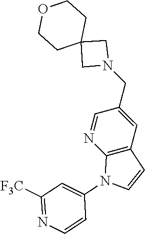 [SMILES: FC(F)(F)c1cc(-n2ccc3cc(CN4CC5(CCOCC5)C4)cnc32)ccn1] | 47 | 2-((1-(2- (Trifluoromethyl) 53yridine-4-yl)- 1H-pyrrolo[2,3- b]53yridine-5- yl)methyl)- 7-oxa-2- azaspiro[3.5] nonane | 1.97 | 402.9 | B |
| 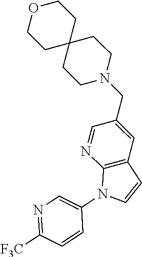 [SMILES: FC(F)(F)c1ccc(-n2ccc3cc(CN4CCC5(CCOCC5)CC4)cnc32)cn1] | 48 | 9-((1-(6- (Trifluoromethyl) pyridin- 3-yl)-1H- pyrrolo[2,3- b]pyridin-5-yl) methyl)-3- oxa-9- azaspiro[5.5] undecane | 2.25 | 431.0 | B |
| 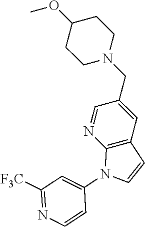 [SMILES: COC1CCN(Cc2cnc3c(ccn3-c3ccnc(C(F)(F)F)c3)c2)CC1] | 49 | 5-((4- Methoxypiperidin- 1-yl)methyl)-1-(2- (trifluoromethyl) pyridin-4-yl)- 1H-pyrrolo[2,3- b]pyridine | 2.17 | 390.9 | B |
| 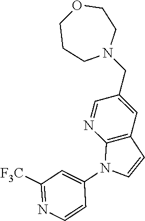 [SMILES: FC(F)(F)c1cc(-n2ccc3cc(CN4CCCOCC4)cnc32)ccn1] | 50 | 4-((1-(2- (Trifluoromethyl) pyridin- 4-yl)-1H-pyrrolo [2,3- b]pyridin-5- yl)methyl)- 1,4-oxazepane | 2.10 | 376.9 | B |

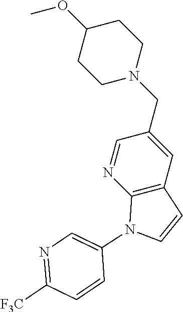 [SMILES: COC1CCN(Cc2cnc3c(ccn3-c3ccc(C(F)(F)F)nc3)c2)CC1]

### Example 51

5-((4-Methoxypiperidin-1-yl)methyl)-1-(6-(trifluoromethyl)pyridin-3-yl)-1H-pyrrolo[2,3-b]pyridine
Step a. Potassium ((4-methoxypiperidin-1-yl)methyl)trifluoroborate

Starting from 4-methoxypiperidine (250 mg, 2.17 mmol) and following the experimental procedure described in step e of Example 28, the title compound was obtained (655 mg, Yield: Quant).

¹H NMR (400 MHZ, CD₃OD) δ 3.56-3.49 (m, 2H), 3.48-3.36 and 3.35-3.33 (2m, 1H), 3.36 and 3.34 (2s, 3H), 3.05 and 2.91 (2t, J=12.4 and 11.9 Hz, 2H), 2.22-1.96 (m, 4H), 1.93-1.79 and 1.72-1.49 (m, 2H).

### Step b. 5-((4-Methoxypiperidin-1-yl)methyl)-1H-pyrrolo[2,3-b]pyridine

Starting from the product obtained in step a (655 mg, 2.17 mmol) and 5-bromo-1H-pyrrolo[2,3-b]pyridine (366 mg, 1.86 mmol) and following the experimental procedure described in step f of Example 28, the title compound was obtained (278 mg, Yield: 61%).

HPLC-MS (Method B): Rt. 1.26 min; ESI⁺-MS m/z: 245.9 (M+1).

Step c. Title Compound

A solution of (6-(trifluoromethyl)pyridin-3-yl)boronic acid (71 mg, 0.37 mmol), the compound obtained in step b (73 mg, 0.25 mmol), Cu(OAc)₂ (45 mg, 0.25 mmol) and TEA (69 μL, 0.5 mmol) in DCE was stirred at rt during 16 h. The reaction was quenched with aq sat NaHCO₃ and the residue was diluted with DCM and washed with aq sat NaHCO₃. The organic layer was dried over anh Na₂SO₄, filtered and concentrated to dryness. The crude product was purified by flash chromatography, silica gel, DCM:MeOH to give the title compound (72 mg, Yield: 74%).

HPLC-MS (Method A): Rt. 2.17 min; ESI⁺-MS m/z: 390.9 (M+1).

This method was used for the preparation of Example 52 using suitable starting materials:

| | | | | MS | |
|---|---|---|---|---|---|
| | | CHEMICAL | Rt | (M + | HPLC |
| STRUCTURE | EX | NAME | (min) | H) | Method |
| 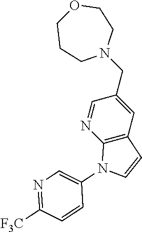 [SMILES: FC(F)(F)c1ccc(-n2ccc3cc(CN4CCCOCC4)cnc32)cn1] | 52 | 4-((1-(6- (Trifluoromethyl) pyridin- 3-yl)-1H- pyrrolo[2,3- b]pyridin-5- yl)methyl)- 1,4-oxazepane | 2.12 | 376.9 | B |

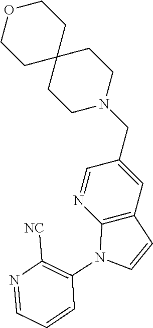 [SMILES: N#Cc1ncccc1-n1ccc2cc(CN3CCC4(CCOCC4)CC3)cnc21]

### Example 53

3-(5-((3-Oxa-9-azaspiro[5.5]undecan-9-yl)methyl)-1H-pyrrolo[2,3-b]pyridin-1-yl) picolinonitrile
Step a. Potassium ((3-oxa-9-azaspiro[5.5]undecan-1-yl)methyl)trifluoroborate

Starting from 3-oxa-9-azaspiro[5.5]undecane (400 mg, 2.57 mmol) and following the experimental procedure described in step e of Example 28, the title compound was obtained (829 mg, Yield: Quant).

¹H NMR (400 MHZ, CD₃OD) δ 3.72-3.62 (m, 4H), 3.39-3.32 (m, 2H), 3.11-2.94 (m, 2H), 2.18-2.10 (m, 2H), 1.98-1.90 (m, 2H), 1.71-1.65 (m, 2H), 1.60 (td, J=14.7, 4.2 Hz, 2H), 1.50-1.42 (m, 2H).

Step b. 9-((1H-Pyrrolo[2,3-b]pyridin-5-yl)methyl)-3-oxa-9-azaspiro[5.5]undecane

Starting from the product obtained in step a (609 mg, 2.21 mmol) and 5-bromo-1H-pyrrolo[2,3-b]pyridine (290 mg, 1.47 mmol) and following the experimental procedure described in step f of Example 28, the title compound was obtained (278 mg, Yield: 66%).

HPLC-MS (Method B): Rt.1.25 min; ESI⁺-MS m/z: 285.9 (M+1).

Step c. Title Compound

A sealed tube charged with 3-bromopicolinonitrile (124 mg, 0.68 mmol), the compound obtained in step b (95 mg, 0.33 mmol), Pd₂(dba)₃ (21 mg, 0.023 mmol), XantPhos (39 mg, 0.068 mmol) and Cs₂CO₃ (147 mg, 0.45 mmol), was evacuated and backfilled with argon. Dioxane (5 mL), degassed by means of bubbling argon to the solution for 5 min, was added and the reaction mixture was stirred at 110° C.overnight. The resulting mixture was filtered through Celite® and washed with EtOAc. The solution was diluted with aq sat NaHCO₃ and extracted twice with EtOAc. The combined organic fractions were dried over anh Na₂SO₄, filtered and concentrated to dryness The solvent was removed under vacuum and the residue was purified by flash chromatography, silica gel, DCM:MeOH to give the title compound (43 mg, Yield: 34%).

HPLC-MS (Method A): Rt. 1.69 min; ESI⁺-MS m/z: 388.2 (M+1).

This method was used for the preparation of Examples 54-56 using suitable starting materials:

| | | CHEMICAL | Rt | MS | HPLC |
|---|---|---|---|---|---|
| STRUCTURE | EX | NAME | (min) | (M + H) | Method |
| 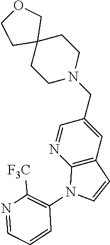 [SMILES: FC(F)(F)c1ncccc1-n1ccc2cc(CN3CCC4(CCOC4)CC3)cnc21] | 54 | 8-((1-(2- (Trifluoromethyl) pyridin- 3-yl)-1H-pyrrolo [2,3- b]pyridin-5-yl) methyl)-2- oxa-8- azaspiro[4.5] decane | 1.78 | 416.9 | B |
| 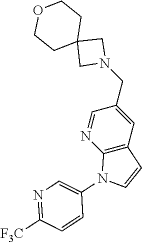 [SMILES: FC(F)(F)c1ccc(-n2ccc3cc(CN4CC5(CCOCC5)C4)cnc32)cn1] | 55 | 2-((1-(6- (Trifluoromethyl) pyridin-3-yl)- 1H-pyrrolo[2,3- b]pyridin-5-yl) methyl)-7- oxa-2- azaspiro[3.5] nonane | 1.98 | 402.9 | B |
| 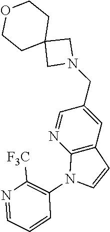 [SMILES: FC(F)(F)c1ncccc1-n1ccc2cc(CN3CC4(CCOCC4)C3)cnc21] | 56 | 2-((1-(2- (Trifluoromethyl) pyridin-3-yl)- 1H-pyrrolo[2,3- b]pyridin-5-yl) methyl)-7- oxa-2- azaspiro[3.5] nonane | 1.63 | 402.9 | B |

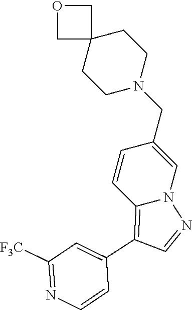 [SMILES: FC(F)(F)c1cc(-c2cnn3cc(CN4CCC5(CC4)COC5)ccc23)ccn1]

### Example 57

7-(3-2-(Trifluoromethyl)pyridin-4-yl)pyrazolo[1,5-a]pyridin-6-yl) methyl)-2-oxa-7-azaspiro[3.5]nonane

### Step a. 6-Methoxy-3-(2-(trifluoromethyl)pyridin-4-yl)pyrazolo[1,5-a]pyridine

A solution of 3-bromo-6-methoxypyrazolo[1,5-a]pyridine (550 mg, 2.42 mmol), 5-(4,4,5,5-tetramethyl-1,3,2-dioxaborolan-2-yl)-2-(trifluoromethyl)pyridine (727 mg, 2.66 mmol), Pd(dppf)₂Cl₂ (177 mg, 0.24 mmol) and Cs₂CO₃ (2.37 g, 7.2 mmol) in dioxane under Ar atmosphere was heated at 110° C. for 16 h. After this time, the solvent was removed under reduced pressure and the residue was diluted with EtOAc and washed with aq sat NaHCO₃. The organic layer was dried over anh Na₂SO₄, filtered and concentrated to dryness. The crude product was purified by flash chromatography, silica gel, DCM:MeOH to give the title compound (437 mg, Yield: 61%).

HPLC-MS (Method B): Rt. 1.95 min; ESI⁺-MS m/z: 294.0 (M+1).

### Step b. 3-(2-(Trifluoromethyl)pyridin-4-yl)pyrazolo[1,5-a]pyridin-6-ol

A 1 M solution of BBr₃ in DCM (3.5 mL, 3.5 mmol) was added to a solution of the product obtained in step a (417 mg, 1.42 mmol) in DCM (5.5 mL) at 0° C. and the solution was stirred at 40° C. for 4 days. The resulting mixture was diluted with DCM and washed with aq sat NaHCO₃. The organic layer was dried over anh Na₂SO₄, filtered and concentrated to dryness. The crude product was purified by flash chromatography, silica gel, DCM:MeOH to give the title compound (347 mg, Yield: 87%).

HPLC-MS (Method B): Rt. 1.64 min; ESI⁺-MS m/z: 280.0 (M+1).

Step c. 3-(2-(Trifluoromethyl)pyridin-4-yl)pyrazolo[1,5-a]pyridin-6-yl trifluoromethanesulfonate

A 1 M solution of Tf₂O in DCM (0.37 mL, 0.37 mmol) was added to a solution of the product obtained in step a (75 mg, 0.27 mmol) and TEA (0.056 mL, 0.4 mmol) in DCM (5.5 mL) at 0° C. and the solution was stirred for 90 min. The resulting mixture was diluted with DCM and washed with aq sat NH₄Cl. The organic layer was washed with brine, dried over anh Na₂SO₄, filtered and concentrated to dryness to give the title compound (58 mg, Yield: 52%).

HPLC-MS (Method A): Rt. 3.24 min; ESI⁺-MS m/z: 412.0 (M+1).

Step d. Title Compound

Starting from the product obtained in step c (58 mg, 0.14 mmol) and the product obtained in step c of Example 29 (38 mg, 0.18 mmol) and following the experimental procedure described in step f of Example 21, the title compound was obtained (6.6 mg, Yield: 12%).

HPLC-MS (Method A): Rt. 1.91 min; ESI⁺-MS m/z: 403.0 (M+1).

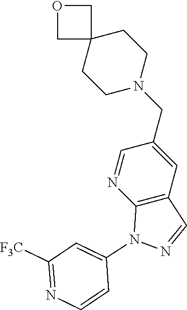 [SMILES: FC(F)(F)c1cc(-n2ncc3cc(CN4CCC5(CC4)COC5)cnc32)ccn1]

### Example 58

7-((1-(2-(Trifluoromethyl)pyridin-4-yl)-1H-pyrazolo[3,4-b]pyridin-5-yl) methyl)-2-oxa-7-azaspiro[3.5]nonane

### Step a. 5-Bromo-1-(2-(trifluoromethyl)pyridin-4-yl)-1H-pyrazolo[3,4-b]pyridine

NaH (60% in mineral oil, 24 mg, 0.6 mmol) was added to a solution of 5-bromo-1H-pyrazolo[3,4-b]pyridine (100 mg, 0.5 mmol) in DMF (3.3 mL) at 0° C. and the suspension was stirred for 15 min. After this time, 4-bromo-2-(trifluoromethyl)pyridine (171 mg, 0.75 mmol) was added and the solution was stirred at 100° C. for 16 h. The resulting mixture was diluted with EtOAc and washed with aq sat NaHCO₃. The organic layer was washed with brine, dried over anh Na₂SO₄, filtered and concentrated to dryness. The crude product was purified by flash chromatography, silica gel, DCM:MeOH, to give the title compound (28 mg, Yield: 16%).

HPLC-MS (Method A): Rt. 2.46 min; ESI⁺-MS m/z: 342.9 (M+1).

Step b. Title Compound

Starting from the product obtained in step a (28 mg, 0.08 mmol) and the product obtained in step c of Example 37 (22 mg, 0.1 mmol) and following the experimental procedure described in step f of Example 28, the title compound was obtained (7.8 mg, Yield: 24%).

HPLC-MS (Method A): Rt. 2.15 min; ESI⁺-MS m/z: 283.0 (M+1).

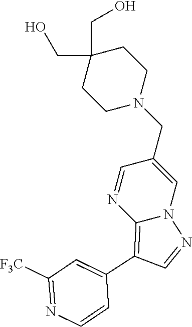 [SMILES: OCC1(CO)CCN(Cc2cnc3c(-c4ccnc(C(F)(F)F)c4)cnn3c2)CC1]

### Example 59

(1-((3-(2-(Trifluoromethyl)pyridin-4-yl)pyrazolo[1,5-a]pyrimidin-6-yl) methyl)piperidine-4,4-diyl)dimethanol

### Method 1

A 2 M aq solution of H₂SO₄ (3.1 mL, 6.2 mmol) was added to a solution of the compound obtained in Example 9 (500 mg, 1.24 mmol) in ACN (15 mL) and the solution was stirred at r.t. for 4 days. The resulting mixture was diluted with EtOAc and washed with aq sat NaHCO₃. The organic layer was dried over anh Na₂SO₄, filtered and concentrated to dryness. The crude product was purified by flash chromatography, silica gel, DCM:MeOH, to give the title compound (166 mg, Yield: 32%).

HPLC-MS (Method B): Rt.1.45 min; ESI⁺-MS m/z: 422.2 (M+1).

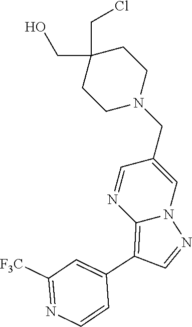 [SMILES: OCC1(CCl)CCN(Cc2cnc3c(-c4ccnc(C(F)(F)F)c4)cnn3c2)CC1]

### Example 60

(4-(Chloromethyl)-1-((3-(2-(trifluoromethyl)pyridin-4-yl)pyrazolo[1,5-a]pyrimidin-6-yl)methyl)piperidin-4-yl)methanol

A 2.5 M solution of HCl in EtOH (0.3 mL, 0.75 mmol) was added to a solution of the product obtained in Example 9 (104 mg, 0.26 mmol) in ACN:H₂O (15:2, 1.7 mL) and the solution was stirred at rt for 1 h. Solvent was removed under reduced pressure and the residue was purified by SCX chromatography (SCX, Agilent bond column). The column was washed with MeOH and then the crude, dissolved in minimal amount of MeOH, loaded onto the SCX column. The column was first eluted with methanol and then with 2 N NH₃ in MeOH. The residue was further purified by flash chromatography, silica gel, DCM:MeOH, to give the title compound (38 mg, Yield: 35%).

HPLC-MS (Method A): Rt. 1.99 min; ESI⁺-MS m/z: 440.0 (M+1).

### Biological Activity

### Pharmacological Study

This invention is aimed at providing a series of compounds which show pharmacological activity towards the σ₁ receptor and/or σ₂ receptor and, especially, compounds which have a binding expressed as Kᵢ responding to the following scales:

Kᵢ (σ₁) is preferably <1000 nM, more preferably <500 nM, even more preferably <100 nM; and

Kᵢ (σ₂) is preferably <1000 nM, more preferably <500 nM, even more preferably <100 nM.

### Human σ₁ Receptor Radioligand Assay

Transfected HEK-293 membranes (7 μg) were incubated with 5 nM of [³H](+)-pentazocine in assay buffer containing Tris-HCl 50 mM at pH 8. NBS (non-specific binding) was measured by adding 10 μM haloperidol. The binding of the test compound was measured at either one concentration (% inhibition at 1 or 10 μM) or five different concentrations to determine affinity values (Ki). Plates were incubated at 37° C. for 120 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail.

### Binding Assay to Human ρ2/TMEM97 Receptor

Transfected HEK-293 membranes (15 μg) were incubated with 10 nM [³H]-1,3-Di-o-tolylguanidine ( DTG) in assay buffer containing Tris-HCl 50 mM at pH 8.0. NSB (non-specific binding) was measured by adding 10 μM haloperidol. The binding of the test compound was measured at either one concentration (% inhibition at 1 or 10 μM) or five different concentrations to determine affinity values (Ki). Plates were incubated at 25° C. for 120 minutes. After the incubation period, the reaction mix was transferred to MultiScreen HTS, FC plates (Millipore), filtered and washed 3 times with ice-cold 10 mM Tris-HCL (pH 8.0). Filters were dried and counted at approximately 40% efficiency in a MicroBeta scintillation counter (Perkin-Elmer) using EcoScint liquid scintillation cocktail.

### Results

The following scale has been adopted for representing the binding to σ1-receptor expressed as Kᵢ:

+Kᵢ (σ₁)>1000 nM or inhibition ranges between 1% and 50%

++500 nM<=Kᵢ(σ₁)<=1000 nM

+++100 nM 21 =Kᵢ(σ₁)<=500 nM

++++Kᵢ(σ₁)<100 nM

The following scale has been adopted for representing the binding to σ₂-receptor expressed as Kᵢ:

+Kᵢ(σ₂) >1000 nM or inhibition ranges between 1% and 50%

++500 nM<=Kᵢ(σ₂)<=1000 nM

+++100 nM<=Kᵢ(σ₂)<=500 nM

++++Kᵢ(σ₂)<100 nM

The binding results of the example compounds for the σ₁ and/or σ₂ receptor are shown in Table 1:

TABLE 1

| Example | Binding σ-1 | Binding σ-2 |
|---|---|---|
| 1 | ++++ | + |
| 2 | +++ | + |
| 3 | ++++ | + |
| 4 | ++++ | + |
| 5 | ++++ | + |
| 6 | ++++ | +++ |
| 7 | +++ | + |
| 8 | ++ | + |
| 9 | ++++ | + |
| 10 | ++++ | + |
| 11 | ++ | + |
| 12 | ++++ | + |
| 13 | ++++ | ++ |
| 14 | ++++ | ++ |
| 15 | ++++ | + |
| 16 | ++++ | + |
| 17 | ++++ | + |
| 18 | ++ | + |
| 19 | ++++ | + |
| 20 | +++ | ++ |
| 21 | ++++ | + |
| 22 | ++++ | + |
| 23 | +++ | + |
| 24 | +++ | + |
| 25 | ++++ | + |
| 26 | +++ | + |
| 27 | ++++ | + |
| 28 | ++++ | ++ |
| 29 | + | + |
| 30 | ++++ | ++ |
| 31 | ++++ | +++ |
| 32 | ++++ | +++ |
| 33 | ++++ | + |
| 34 | ++++ | + |
| 35 | ++ | + |
| 36 | ++++ | + |
| 37 | ++++ | ++ |
| 38 | ++++ | +++ |
| 39 | ++++ | +++ |
| 40 | ++++ | +++ |
| 41 | ++++ | +++ |
| 42 | ++++ | +++ |
| 43 | ++++ | +++ |
| 44 | ++++ | +++ |
| 45 | ++++ | +++ |
| 46 | ++++ | ++++ |
| 47 | ++++ | ++++ |
| 48 | ++++ | +++ |
| 49 | ++++ | ++ |
| 50 | ++++ | + |
| 51 | ++++ | ++ |
| 52 | ++++ | ++ |
| 53 | ++++ | ++ |
| 54 | ++++ | ++ |
| 55 | ++++ | +++ |
| 56 | ++++ | + |
| 57 | ++++ | +++ |
| 58 | ++++ | +++ |
| 59 | ++++ | + |
| 60 | ++++ | +++ |

1. A compound of general formula (I′): 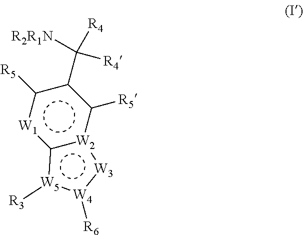
wherein:
W₁, W₂, W₃, W₄ and W₅ represent independently from one another a N atom, a C atom or a —CH— with the proviso that irrespective of their meaning the rings are always aromatic as represented by the dotted lines;
R₁ and R₂ are independently from one another hydrogen or a linear or branched C₁-C₆ alkyl radical or, alternatively, R₁ and R₂ together with the N atom to which they are attached, form a saturated heterocycle optionally containing a further heteroatom selected from N or O and optionally substituted by at least one of a C₁-C₆ alkyl radical, a halogen atom, a C₁-C₆ haloalkyl radical, an —(CH₂)ₙOR group, an aryl group, a 3 to 6 membered saturated heterocycle containing at least one heteroatom selected from Nor O, or alternatively R₁ and R₂ together with the N atom to which they are attached, form a saturated spiro-heterocycle containing optionally an additional heteroatom selected from O, N or S and being optionally substituted by at least one of a C₁-C₆ alkyl radical, a halogen atom, a C₁-C₆ haloalkyl radical, an —(CH₂)ₙOR group;
R is a H atom or an C₁-C₆ alkyl radical;
n is 0 or 1;
R₃ is a pyridinyl radical optionally substituted by a C₁-C₆ alkyl radical, a halogen atom, a C₁-C₆ haloalkyl radical, a OH group, a C₁₋₆ alkoxy group or a cyano group;
R₄, R₄′ are independently from one another hydrogen or a linear or branched C₁-C₆ alkyl radical;
R₅ and R₅′ are independently from one another hydrogen, halogen, C₁₋₆-alkyl, C₁₋₆-haloalkyl, C₁₋₆ alkoxy, —CN; and
R₆ is hydrogen, halogen, C₁₋₆-alkyl, C₁₋₆-haloalkyl, C₁₋₆ alkoxy, —CN, wherein the compound of formula (I′) is optionally in form of one of the stereoisomers, a racemate or in form of a mixture of at least two of the stereoisomers, in any mixing ratio, or a corresponding salt, co-crystal or prodrug thereof.

2. A compound according to claim 1, wherein:
R₁ and R₂ are independently from one another, C₁-C₆ alkyl radical, preferably ethyl, propyl or isopropyl; or
R₁ and R₂ together with the N atom to which they are attached form a pyrrolidine ring; a morpholine ring; an oxazepane ring; a piperazine ring optionally substituted by an oxetanyl; or piperidine ring radical, optionally substituted with a group selected from a phenyl radical, at least one halogen atom and a —(CH₂)ₙOR group; or
R₁ and R₂ together with the N atom form an oxa-azaspiro alkane, preferably oxa-azaspiro-nonane, oxa-azaspiro-decane or oxa-azaspiro-undecane.

3. A compound according to claim 1, wherein R₃ is a pyridinyl radical optionally substituted with halogen, C₁₋₆-alkyl, C₁₋₆-haloalkyl or C₁₋₆ alkoxy.

4. A compound according to claim 1, wherein R₄, R₄′, R₅, R₅′ and R₆ is H.

5. A compound according to claim 1 having one of the general formulae (I), (I′a), (I′b) or (I′c):

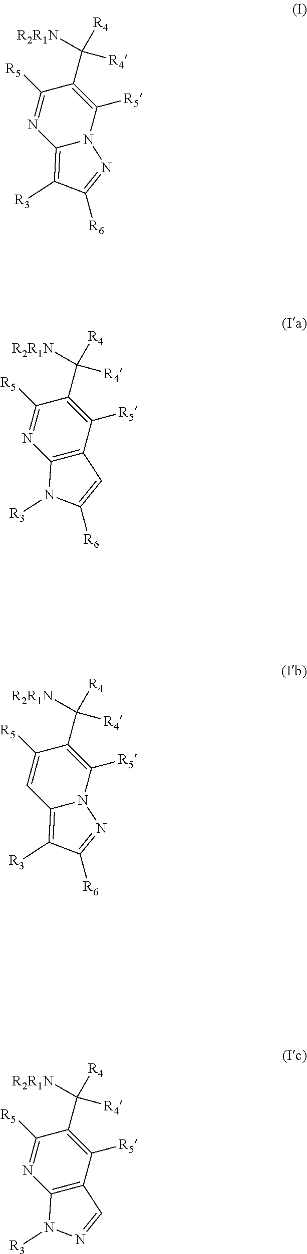

6. A compound according to claim 1 having the general formula (I):

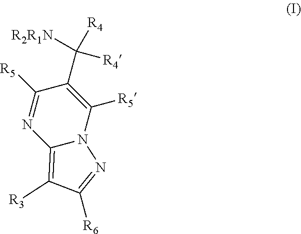

7. A compound according to claim 1 selected from the following list:
[1] 6-(Pyrrolidin-1-ylmethyl)-3-(2-(trifluoromethyl)pyridin-4-yl)pyrazolo[1,5-a]pyrimidine;
[2] 3-(Pyridin-4-yl)-6-(pyrrolidin-1-ylmethyl)pyrazolo[1,5-a]pyrimidine;
[3] 3-(3-Fluoropyridin-4-yl)-6-(pyrrolidin-1-ylmethyl)pyrazolo[1,5-a]pyrimidine;
[4] 3-(2-Methoxypyridin-3-yl)-6-(pyrrolidin-1-ylmethyl)pyrazolo[1,5-a]pyrimidine;
[5] 6-((4-Phenylpiperidin-1-yl)methyl)-3-(pyridin-4-yl)pyrazolo[1,5-a]pyrimidine;
[6] 6-((4-Phenylpiperidin-1-yl)methyl)-3-(2-(trifluoromethyl)pyridin-4 y1)pyrazolo[1,5-a]pyrimidine;
[7] 7-((3-(2-(Trifluoromethyl)pyridin-4-yl)pyrazolo[1,5-a]pyrimidin-6-yl)methyl)-1-oxa-7-azaspiro[3.5]nonane;
[8] 7-((3-(3-Fluoropyridin-4-yl)pyrazolo[1,5-a]pyrimidin-6-yl)methyl)-1-oxa-7-azaspiro[3.5]nonane;
[9] 7-((3-(2-(Trifluoromethyl)pyridin-4-yl)pyrazolo[1,5-a]pyrimidin-6-yl)methyl)-2-oxa-7-azaspiro[3.5]nonane;
[10] 8-((3-(2-(Trifluoromethyl)pyridin-4-yl)pyrazolo[1,5-a]pyrimidin-6-yl)methyl)-2-oxa-8-azaspiro[4.5]decane;
[11] 6-((4,4-Difluoropiperidin-1-yl)methyl)-3-(2-(trifluoromethyl)pyridin-4-yl) pyrazolo[1,5-a]pyrimidine;
[12] N-Ethyl-N-((3-(2-(trifluoromethyl)pyridin-4-yl)pyrazolo[1,5-a]pyrimidin-6-yl) methyl)ethanamine;
[13] 7-((3-(6-(Trifluoromethyl)pyridin-3-yl)pyrazolo[1,5-a]pyrimidin-6-yl)methyl)-2-oxa-7-azaspiro[3.5]nonane;
[14] 7-((3-(6-(Trifluoromethyl)pyridin-2-yl)pyrazolo[1,5-a]pyrimidin-6-yl)methyl)-2-oxa-7-azaspiro[3.5]nonane;
[15] 7-((3-(2-Methylpyridin-4-yl)pyrazolo[1,5-a]pyrimidin-6-yl)methyl)-2-oxa-7-azaspiro[3.5]nonane;
[16] 7-((3-(2-Chloropyridin-4-yl)pyrazolo[1,5-a]pyrimidin-6-yl)methyl)-2-oxa-7-azaspiro[3.5]nonane;
[17] 7-((3-(4-(Trifluoromethyl)pyridin-3-yl)pyrazolo[1,5-a]pyrimidin-6-yl)methyl)-2-oxa-7-azaspiro[3.5]nonane;
[18] 7-((3-(Pyridin-2-yl)pyrazolo[1,5-a]pyrimidin-6-yl)methyl)-2-oxa-7-azaspiro[3.5]nonane;
[19] 7-((3-(3-(Trifluoromethyl)pyridin-4-yl)pyrazolo[1,5-a]pyrimidin-6-yl)methyl)-2-oxa-7-azaspiro[3.5]nonane;
[20] 7-((2-Methyl-3-(2-(trifluoromethyl)pyridin-4-yl)pyrazolo[1,5-a]pyrimidin-6-yl) methyl)-2-oxa-7-azaspiro[3.5]nonane;
[21] 6-((4-Methoxypiperidin-1-yl)methyl)-3-(6-(trifluoromethyl)pyridin-3-yl) pyrazolo[1,5-a]pyrimidine;
[22] 2-((3-(6-(Trifluoromethyl)pyridin-3-yl)pyrazolo[1,5-a]pyrimidin-6-yl)methyl)-7-oxa-2-azaspiro[3.5]nonane;
[23] 6-((4-Methoxypiperidin-1-yl)methyl)-3-(2-methylpyridin-4-yl)pyrazolo[1,5-a]pyrimidine;
[24] 4-((3-(2-Methylpyridin-4-yl)pyrazolo[1,5-a]pyrimidin-6-yl)methyl)-1,4-oxazepane;
[25] 6-((4-(Methoxymethyl)piperidin-1-yl)methyl)-3-(2-methylpyridin-4-yl)pyrazolo[1,5-a]pyrimidine;
[26] 9-((3-(2-Methylpyridin-4-yl)pyrazolo[1,5-a]pyrimidin-6-yl)methyl)-3-oxa-9-azaspiro[5.5]undecane;
[27] 2-((3-(2-Methylpyridin-4-yl)pyrazolo[1,5-a]pyrimidin-6-yl)methyl)-7-oxa-2-azaspiro[3.5]nonane;
[28] 6-((4-(Methoxymethyl)piperidin-1-yl)methyl)-3-(2-(trifluoromethyl)pyridin-4-yl)pyrazolo[1,5-a]pyrimidine;
[29] 4-((3-(2-(Trifluoromethyl)pyridin-4-yl)pyrazolo[1,5-a]pyrimidin-6-yl) methyl)morpholine;
[30] 6-((4-(Ethoxymethyl)piperidin-1-yl)methyl)-3-(2-(trifluoromethyl)pyridin-4-yl)pyrazolo[1,5-a]pyrimidine;
[31] 4-((3-(2-(Trifluoromethyl)pyridin-4-yl)pyrazolo[1,5-a]pyrimidin-6-yl)methyl)-1,4-oxazepane;
[32] 9-((3-(2-(Trifluoromethyl)pyridin-4-yl)pyrazolo[1,5-a]pyrimidin-6-yl)methyl)-3-oxa-9-azaspiro[5.5]undecane;
[33] 2-((3-(2-(Trifluoromethyl)pyridin-4-yl)pyrazolo[1,5-a]pyrimidin-6-yl)methyl)-7-oxa-2-azaspiro[3.5]nonane;
[34] 6-((4-Methoxypiperidin-1-yl)methyl)-3-(2-(trifluoromethyl)pyridin-4-yl)pyrazolo[1,5-a]pyrimidine;
[35] 6-((4-(Oxetan-3-yl)piperazin-1-yl)methyl)-3-(2-(trifluoromethyl)pyridin-4-yl) pyrazolo[1,5-a]pyrimidine;
[36] 1-((3-(2-(Trifluoromethyl)pyridin-4-yl)pyrazolo[1,5-a]pyrimidin-6-yl) methyl)piperidin-4-ol;
[37] 7-((5,7-Dimethyl-3-(2-(trifluoromethyl)pyridin-4-yl)pyrazolo[1,5-a]pyrimidin-6-yl) methyl)-2-oxa-7-azaspiro[3.5]nonane;
[38] 7-((1-(2-(Trifluoromethyl)pyridin-4-yl)-1H-pyrrolo[2,3-b]pyridin-5-yl)methyl)-2-oxa-7-azaspiro[3.5]nonane;
[39] 5-((4-(Methoxymethyl)piperidin-1-yl)methyl)-1-(2-(trifluoromethyl)pyridin-4-yl)-1H-pyrrolo[2,3-b]pyridine;
[40] 5-((4-(Ethoxymethyl)piperidin-1-yl)methyl)-1-(2-(trifluoromethyl)pyridin-4-yl)-1H-pyrrolo[2,3-b]pyridine;
[41] 5-((4-(Methoxymethyl)piperidin-1-yl)methyl)-1-(6-(trifluoromethyl)pyridin-3-yl)-1H-pyrrolo[2,3-b]pyridine;
[42] 7-((1-(6-(Trifluoromethyl)pyridin-3-yl)-1H-pyrrolo[2,3-b]pyridin-5-yl)methyl)-2-oxa-7-azaspiro[3.5]nonane;
[43] 8-((1-(2-(Trifluoromethyl)pyridin-4-yl)-1H-pyrrolo[2,3-b]pyridin-5-yl)methyl)-2-oxa-8-azaspiro[4.5]decane;
[44] 5-((4-(Methoxymethyl)piperidin-1-yl)methyl)-1-(2-(trifluoromethyl)pyridin-3-yl)-1H-pyrrolo[2,3-b]pyridine;
[45] 9-((1-(2-(Trifluoromethyl)pyridin-3-yl)-1H-pyrrolo[2,3-b]pyridin-5-yl)methyl)-3-oxa-9-azaspiro[5.5]undecane;
[46] 9-((1-(2-(Trifluoromethyl)pyridin-4-yl)-1H-pyrrolo[2,3-b]pyridin-5-yl)methyl)-3-oxa-9-azaspiro[5.5]undecane;
[47] 2-((1-(2-(Trifluoromethyl)pyridine-4-yl)-1H-pyrrolo[2,3-b]pyridine-5-yl)methyl)-7-oxa-2-azaspiro[3.5]nonane;
[48] 9-((1-(6-(Trifluoromethyl)pyridin-3-yl)-1H-pyrrolo[2,3-b]pyridin-5-yl)methyl)-3-oxa-9-azaspiro[5.5]undecane;
[49] 5-((4-Methoxypiperidin-1-yl)methyl)-1-(2-(trifluoromethyl)pyridin-4-yl)-1H-pyrrolo[2,3-b]pyridine;
[50] 4-((1-(2-(Trifluoromethyl)pyridin-4-yl)-1H-pyrrolo[2,3-b]pyridin-5-yl)methyl)-1,4-oxazepane;
[51] 5-((4-Methoxypiperidin-1-yl)methyl)-1-(6-(trifluoromethyl)pyridin-3-yl)-1H-pyrrolo[2,3-b]pyridine;
[52] -((1-(6-(Trifluoromethyl)pyridin-3-yl)-1H-pyrrolo[2,3-b]pyridin-5-yl)methyl)-1,4-oxazepane;
[53] 3-(5-((3-Oxa-9-azaspiro[5.5]undecan-9-yl)methyl)-1H-pyrrolo[2,3-b]pyridin-1-yl) picolinonitrile;
[54] 8-((1-(2-(Trifluoromethyl)pyridin-3-yl)-1H-pyrrolo[2,3-b]pyridin-5-yl)methyl)-2-oxa-8-azaspiro[4.5]decane;
[55] 2-((1-(6-(Trifluoromethyl)pyridin-3-yl)-1H-pyrrolo[2,3-b]pyridin-5-yl)methyl)-7-oxa-2-azaspiro[3.5]nonane;
[56] 2-((1-(2-(Trifluoromethyl)pyridin-3-yl)-1H-pyrrolo[2,3-b]pyridin-5-yl)methyl)-7-oxa-2-azaspiro[3.5]nonane;
[57] 7-((3-(2-(Trifluoromethyl)pyridin-4-yl)pyrazolo[1,5-a]pyridin-6-yl)methyl)-2-oxa-7-azaspiro[3.5]nonane;
[58] 7-((1-(2-(Trifluoromethyl)pyridin-4-yl)-1H-pyrazolo[3,4-b]pyridin-5-yl)methyl)-2-oxa-7-azaspiro[3.5]nonane;
[59] (1-((3-(2-(Trifluoromethyl)pyridin-4-yl)pyrazolo[1,5-a]pyrimidin-6-yl) methyl)piperidine-4,4-diyl)dimethanol; and
[60] (4-(Chloromethyl)-1-((3-(2-(trifluoromethyl)pyridin-4-yl)pyrazolo[1,5-a]pyrimidin-6-yl) methyl)piperidin-4-yl)methanol.

8. A process Process for the preparation of a compound of formula (I): 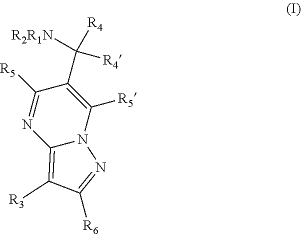
comprising:
a reaction of a compound of formula (VI): 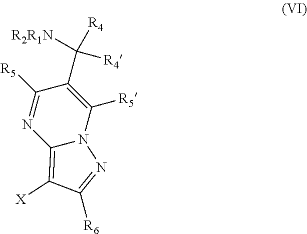
with a compound of formula (VII): 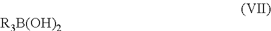
wherein X is a halogen atom.

9. A process for the preparation of the compound of formula (Ia): 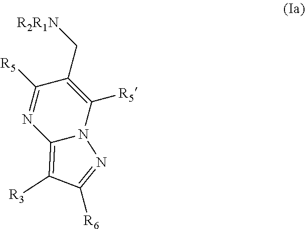
comprising the reaction of a compound of formula (XII): 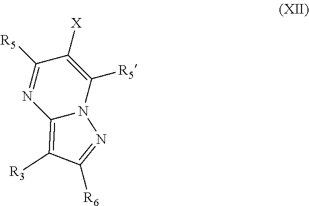
with a compound of formula (XIII): 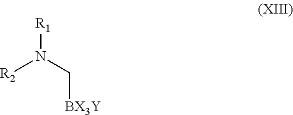
wherein X is a halogen and Y is an alkaline atom.

10. A compound according to claim 1 for use as a medicament.

11. A compound according to claim 10 for use in one or both of the treatment and prophylaxis of one or both of diseases and disorders mediated by a sigma receptor.

12. A compound for use according to claim 11, wherein said sigma receptor is one or both of a sigma-1 receptor and a sigma-2 receptor.

13. A compound for use according to claim 12, wherein the disease or disorder is:
pain, selected from neuropathic pain, inflammatory pain, chronic pain or any other pain conditions involving one or both of allodynia and hyperalgesia; or
a CNS disorder or disease, selected from a group including addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia and paranoia.

14. A pharmaceutical composition comprising:
a compound of general formula (I) according to claim 1 or a pharmaceutically acceptable salt, isomer, co-crystal prodrug or solvate thereof, and at least a pharmaceutically acceptable carrier, additive, adjuvant or vehicle.
